(19) United States
(12) Patent Application Publication
(10) Pub. No.: US 2002/0192796 A1
(43) Pub. Date: Dec. 19, 2002

(54) Anti-sigma28 factors in Helicobacter pylori, Campylobacter jejuni and Pseudomonas aeruginosa and applications thereof

(76) Inventors: Pierre Legrain, Paris (FR); Frederic Colland, Fosses (FR); Jean-Christophe Rain, Puteaux (FR); Agnes Labigne, Bures-sur-yvette (FR); Hilde De Reuse, Paris (FR)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/066,127

(22) Filed: Jan. 31, 2002

Related U.S. Application Data
(60) Provisional application No. 60/265,465, filed on Jan. 31, 2001.

(51) Int. Cl. C12N 9/50; C07H 21/04; C12P 21/02; C12N 1/21

(52) U.S. Cl. 435/219; 435/69.3; 435/320.1; 435/252.3; 536/23.2

(57) ABSTRACT

Disclosed are polypeptides named HP1122, Cj1464 and PA3351 which are the anti-σ²⁸ factor of Helicobacter pylori, Campylobacter jejuni and Pseudomonas aeruginosa, respectively and fragments and variants thereof. Also disclosed is a polypeptide named SID1122 which is the domain of Helicobacter pylori's HP1122 polypeptide involved in a specific interaction with Helicobacter pylori σ²⁸ (HP1032) and which has an anti-σ²⁸ factor activity. Further disclosed are a SID1122 polypeptide that interacts with HP1032, identification of the HP1032 interacting domain (SID1032) that is specifically involved in the interaction with HP1122, complexes of two polypeptides such as HP1122-HP1032, or SID1122-SID1032, fragments and variants of the SID1122 and SID1032 polypeptides, antibodies to the SID1122 and SID1032 polypeptides, methods for screening drugs or agents which modulate the interaction of Helicobacter pylori's polypeptides encoded by HP1122 and HP1032, and pharmaceutical compositions for treating or preventing Gram negative flagellated bacteria infection in a human or mammal, more specifically Helicobacter sp. or Campylobacter jejuni or Pseudomonas aeruginosa infection, in particular Helicobacter pylori infection in a human or a mammal.

### CROSS-REFERENCE TO RELATED APPLICATIONS

The present application claims priority to U.S. provisional application No. 60/265,465 filed Jan. 31, 2001.

### BACKGROUND OF THE INVENTION

Helicobacter pylon (H. pylori) is a microaerophilic, Gram negative, slow growing spiral shaped and flagellated organism. H. pylori has first been isolated in 1984 from a gastric biopsy specimen of a patient with chronic gastritis (Marshall and Warren, 1984).

The organization of the physical structure of the H. pylori flagellum is similar to that of enteric organisms Escherichia coli and Salmonella typhimurium. The flagellum is composed of three structural elements: a basal body, a flexible hook and a flagellar filament. The mechanism of gene expression involved in flagellar assembly was extensively studied in the model organisms mentioned above (for review, see Macnab et al., 1996). In E. coli and S. typhimurium genes involved in flagellar biosynthesis are expressed in a hierarchical order and divided into three classes: (i) the class 1 genes, flhC and flhD, are regulated by the σ⁷⁰ factor of RNA polymerase. The FlhC and FlhD proteins act as transcriptional activators to stimulate transcription from class 2 genes; (ii) the class 2 genes encode the early components required for flagellar assembly such as the basal body and flexible hook (HBB complex) as well as the σ²⁸ factor of RNA polymerase; and (iii) the expression of the class 3 genes, which encode proteins involved in the final stages of flagellar assembly, is controlled by the σ²⁸ factor. Completion of the HBB complex is required to result in class 3 gene expression. This tight regulation is due to the presence of the anti-σ²⁸ factor, FIgM, which binds to σ²⁸ and prevents its association with RNA polymerase core enzyme (Ohnishi et al., 1992; for review, see Hughes & Mathee, 1998). It has been shown that FIgM is secreted from the cell through the HBB structures thus allowing RNA polymerase associated with σ²⁸ to transcribe class 3 genes (Gillen & Hughes, 1991a; 1991b; Hughes et al., 1993; Kutsukake, 1994).

The recent sequencing of the H. pylori strain 26695 suggests the presence of about 40 genes involved in flagellar biosynthesis (Tomb et al., 1997). However, the mechanism of flagellar gene regulation seems to differ from those of other bacteria (for review, see O'Toole et al., 2000). In H. pylori, there is no transcriptional activator such as FlhC or FlhD, which belong to the class 1 genes. In addition, a second σ factor, σ⁵⁴, is involved in regulation of some flagellar genes (Spohn & Scarlato, 1999). For example, the components of flagellar filaments, FlaA and FlaB, are differently regulated (Leying et al., 1992; Suerbaum et al., 1993). Transcription control of flaA, which encode the major subunit, is supposed to be exerted by σ²⁸ (HP1032) whereas flaB expression, which encodes the minor subunit, is controlled by σ⁵⁴. The σ⁵⁴ regulon requires the presence of FlgR, an activator protein of the NtrC family. Finally, no genes encoding proteins with significant homology to the anti-σ²⁸ factor has been identified in H. pylori yet.

Helicobacter pylori is the causative agent associated with gastritis and gastric ulcers and has been associated with some types of gastric cancers (McGowan et al., 1996). Several bacterial factors such as urease (Cussac et al., 1992), CagA encoded by the cytotoxin-associated gene (Covacii et al., 1993; Tummuru et al., 1993), the vacuolating toxin (Cover et al., 1994; Telford et al., 1994) and flagellins (Leying et al., 1992; Haas et al., 1993; Suerbaum et al., 1993) have been suggested to play a role in virulence. In addition, motility has been shown to be of major importance for the colonization ability of H. pylori in the piglet model (Eaton et al., 1992). The motility of H. pylori is considered as one of the major virulence factors. It appears that the flagellum biogenesis could be an ideal target for antimicrobial therapies.

### SUMMARY OF THE PRESENT INVENTION

One aspect of the present invention is directed to isolated and/or purified proteins which are the anti-σ²⁸ factors of Helicobacter Pylori, Campylobacter jejuni and Pseudomonas aeruginosa, and fragments and variants thereof. In one embodiment, selective interacting domains (SID®) of these proteins are provided.

A related aspect of the present invention is directed to polynucleotides encoding the aforementioned polypeptides, pharmaceutical compositions containing the polynucleotides, and methods of using the compositions for therapeutic purposes.

Another aspect of the present invention is directed to complexes between the anti-σ²⁸ factor proteins, or variants or interacting fragments thereof, and their corresponding σ²⁸ factor proteins,or variants or interacting fragments thereof.

Another aspect of the present invention is directed to a method for identifying or screening for modulating compounds (e.g., drugs or agents) that modulate protein-protein interactions involving the σ²⁸ and/or anti-σ²⁸ factors of Helicobacter Pylori, Campylobacter jejuni or Pseudomonas aeruginosa. Also provided in this regard are kits for conducting the methods.

Yet another aspect of the present invention is directed to antibodies, e.g., polyclonal and monoclonal, that recognize the σ²⁸ and/or anti-σ²⁸ factors of these bacteria.

Yet another aspect of the present invention is directed to methods for preventing or treating infection in humans or other mammals by gram-negative flagellated bacteria such as Helicobacter pylori, Campylobacter jejuni or Pseudomonas aeruginosa.

### BRIEF DESCRIPTION OF THE DRAWINGS

FIG. 1 depicts the results of a two-hybrid screening between HP1122 and σ²⁸ (FliA or HP1032). The interaction viewer exhibits the detailed experimental results supporting the interaction between the bait (HP1122) and the peptidic preys generated from HP1032. 36 independent fragments, encoding HP1032, were characterized and the Selected Interacting Domain (SID1032) of σ²⁸ identified (residues 198-255).

FIG. 2 is a schematic representation of the HP1032-HP1122 interaction. Conserved regions of σ²⁸ are represented as defined by Lonetto et al. (1992). SIDs identified in this interaction are indicated: region 4 of σ²⁸ (residues 198-255, SID1032) and SID1122 (residues 48-76).

FIG. 3 shows the growth phenotype of diploid yeast strains containing different plasmids and analyzed by incubating cells at various dilutions (from 1 to 10⁻⁴) (A). Yeast growth was performed during 2 days at 30° C. in the absence of methionine on DO-2 or DO-3 medium. Cells contain [p3H1-σ²⁸]+pP6(β) in lane 1, [p3H1-σ²⁸-HP1122]+pP6(β) in lanes 2 and 3, [p3H1-σ²⁸-SID419]+pP6(β) in lane 4, [p3H1-α-HP1122]+pP6(β) in lane 5, [p3H1-σ²⁸-HP1122]+pP6(HP0875) in lane 6. (B) β-galactosidase assay on yeast cell extracts transformed with plasmids described above and incubated on DO-2 medium. Experiments were performed in triplicate.

FIG. 4 shows the growth phenotype of diploid yeast strains containing different plasmids and analyzed by incubating cells at various dilutions (from 1 to 10⁻⁴) (A). Yeast growth was performed during 2 days at 30° C. in the absence of methionine on DO-2 or DO-3 medium. Cells contain [p3H1-σ²⁸]+pP6(β) in lane 1, [p3H1-σ²⁸-SID1122]+pP6(β) in lanes 2 and 3, [p3H1-σ²⁸-SID419]+pP6(β) in lane 4, [p3H1-α-SID1122]+pP6(β) in lane 5, [p3H1-σ²⁸-SID1122]+pP6(HP0875) in lane 6. (B) β-galactosidase assay on yeast cell extracts transformed with plasmids described above and incubated on DO-2 medium. Experiments were performed in triplicate.

FIG. 5 shows a slot blot hybridization assay of total RNAs with probes specific for flaA mRNA. The probe specific for 16S rRNA was used for normalization. Tests were performed on H. pylori N6 wild type strain (lane 1), isogenic mutant σ²⁸ (HP1032) (lane 2), isogenic mutant HP1122 (lane 3) and HP1122-overexpressing strain (lane 4).

FIG. 6 depicts results of electron microscopy of H. pylori (A) N6 wild type strain, (B) isogenic mutant σ²⁸ (HP1032), (C) HP1122-overexpressing strain and (D) SID1122-overexpressing strain. Grids were examined at ×17.000 magnification.

FIG. 7 shows an alignment between H. pylori HP1122, E. coli FlgM and B. subtilis FlgM. The three sequences were aligned using the FastA algorithm. Conserved residues among three proteins (black) and only two of the three proteins (grey) are outlined. The position of HP1122 C-terminal domain interacting with σ²⁸ is indicated by a line.

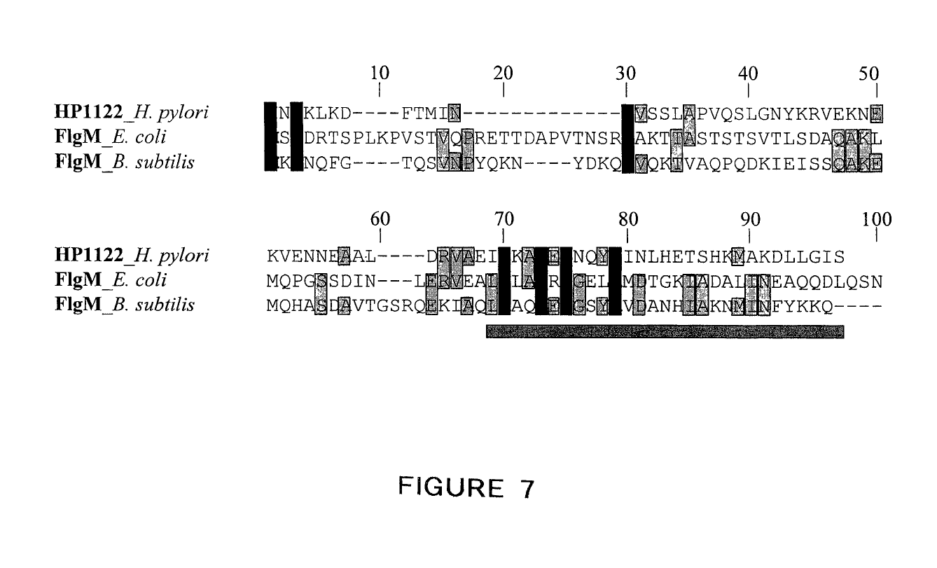

FIG. 8 is a schematic representation of the pP6 plasmid.

FIG. 9 is a schematic representation of the pRH220cat plasmid.

### DETAILED DESCRIPTION OF THE PREFERRED EMBODIMENTS

As used herein the terms “polynucleotides”, “nucleic acids” and “oligonucleotides” are used interchangeably and include, but are not limited to RNA, DNA, RNA/DNA sequences of more than one nucleotide in either single chain or duplex form. The polynucleotide sequences of the present invention may be prepared from any known method including, but not limited to, any synthetic method, any recombinant method, any ex vivo generation method and the like, as well as combinations thereof.

The term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications such as polypeptides having covalent attachment of glycosyl groups, acetyl groups, phosphate groups, lipid groups and the like. Also encompassed by this definition of “polypeptide” are polymer of amino acids encoded by homologs thereof.

By the term “homologs” is meant structurally similar genes contained within a given species, and orthologs or paralogs which are functionally equivalent genes from a given species or strain, as determined for example, in a standard complementation assay. Thus, a polypeptide of interest can be used not only as a model for identifying similar genes in given strains, but also to identify polypeptides of interest encoded by homologs and orthologs or paralogs in other species. The orthologs or paralogs, for example, can also be identified in a conventional complementation assay. In addition or alternatively, such orthologs or paralogs can be expected to exist in bacteria (or other kind of cells) in the same branch of the phylogenic tree, as set forth, for example, at ftp://ftp.cme.msu.edu/Pub/rdp/SSU-rRNA/SSU/Prok.phylo.

As used herein the term “complementary” means that, for example, each base of a first polynucleotide is paired with the complementary base of a second polynucleotide whose orientation is reversed. The complementary bases are A and T (or A and U) or C and G.

The term “sequence identity” refers to the identity between two peptides or between two nucleic acids. Identity between sequences can be determined by comparing a position in each of the sequences which may be aligned for the purposes of comparison. When a position in the compared sequences is occupied by the same base or amino acid, then the sequences are identical at that position. A degree of sequence identity between nucleic acid sequences is a function of the number of identical nucleotides at positions shared by these sequences. A degree of identity between amino acid sequences is a function of the number of identical amino acid sequences that are shared between these sequences. Since two polypeptides may each (i) comprise a sequence (i.e., a portion of a complete polynucleotide sequence) that is similar between two polynucleotides; and (ii) may further comprise a sequence that is divergent between two polynucleotides, sequence identity comparisons between two or more polynucleotides over a “comparison window” refers to the conceptual segment of at least 20 contiguous nucleotide positions wherein a polynucleotide sequence may be compared to a reference nucleotide sequence of at least 20 contiguous nucleotides and wherein the portion of the polynucleotide sequence in the comparison window may comprise additions or deletions (i.e., gaps) of 20 percent or less compared to the reference sequence (which does not comprise additions or deletions) for optimal alignment of the two sequences.

To determine the percent identity of two amino acid sequences or two nucleic acid sequences, the sequences are aligned for optimal comparison. For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position.

The percent identity between the two sequences is a function of the number of identical positions shared by the sequences. Hence % identity=number of identical positions/total number of overlapping positions X 100.

In this comparison the sequences can be the same length or may be different in length. Optimal alignment of sequences for determining a comparison window may be conducted by the local homology algorithm of Smith and Waterman (1981), by the homology alignment algorithm of Needleman and Wunsch (1972), by the search for similarity via the method of Pearson and Lipman (1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection.

The best alignment (i.e., resulting in the highest percentage of identity over the comparison window) generated by the various methods is selected.

The term “sequence identity” means that two polynucleotide sequences are identical (i.e., on a nucleotide by nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size) and multiplying the result by 100 to yield the percentage of sequence identity. The same process can be applied to polypeptide sequences.

The percentage of sequence identity of a nucleic acid sequence or an amino acid sequence can also be calculated using BLAST software (Version 2.06 of September 1998) with the default or user defined parameter.

The term “sequence similarity” means that amino acids can be modified while retaining the same function. It is known that amino acids are classified according to the nature of their side groups and some amino acids such as the basic amino acids can be interchanged for one another while their basic function is maintained.

The term “isolated” as used herein means that a biological material such as a nucleic acid or protein has been removed from its original environment in which it is naturally present. For example, a polynucleotide present in a plant, mammal or animal is present in its natural state and is not considered to be isolated. The same polynucleotide separated from the adjacent nucleic acid sequences in which it is naturally inserted in the genome of the plant or animal is considered as being “isolated.”

The term “isolated” is not meant to exclude artificial or synthetic mixtures with other compounds, or the presence of impurities which do not interfere with the biological activity and which may be present, for example, due to incomplete purification, addition of stabilizers or mixtures with pharmaceutically acceptable excipients and the like.

“Isolated polypeptide” or “isolated protein” as used herein means a polypeptide or protein which is substantially free of those compounds that are normally associated with the polypeptide or protein in a naturally state such as other proteins or polypeptides, nucleic acids, carbohydrates, lipids and the like.

The term “purified” as used herein means at least one order of magnitude of purification is achieved, preferably two or three orders of magnitude, most preferably four or five orders of magnitude of purification of the starting material or of the natural material. Thus, the term “purified” as utilized herein does not mean that the material is 100% purified and thus excludes any other material.

As used herein, the term “antibody” refers to a polypeptide or a group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as well as fragments, including Fab, Fab′, F(ab)2 and F(ab′)2 fragments (see Blum et al., 2000 and Biocca et al., 1990).

By polynucleotides, polypeptides, fragments and variants thereof, it is meant isolated or purified polynucleotides, polypeptides, fragments and variants thereof.

As used herein, “SID®” means a Selected Interacting Domain and is identified as follows: for each bait polypeptide screened, selected prey polypeptides are compared. Overlapping fragments in the same ORF (Open reading Frame) or CDS (coding sequence) define the SID.

The term “variants” when referring to, for example, polynucleotides encoding a polypeptide variant of a given reference polypeptide are polynucleotides that encode a polypeptide that differ from the reference polypeptide by at least one structural or functional characteristic of the reference polypeptide, this polypeptide may maintain functional characteristics of the reference polypeptide with more or less affinity or may have different functional characteristics. A variant of a polynucleotide may be a naturally occurring allelic variant or it may be a variant that is known naturally not to occur. Such non-naturally occurring variants of the reference polynucleotide can be made by, for example, mutagenesis techniques, including those mutagenesis techniques that are applied to polynucleotides, cells or organisms.

Generally, differences are limited so that the nucleotide sequences of the reference and variant are closely similar overall and, in many regions identical.

Variants of polynucleotides according to the present invention include, but are not limited to, nucleotide sequences which are at least 80% identical after alignment to the reference polynucleotide encoding the reference polypeptide. These variants can also have 96%, 97%, 98% and 99.99% sequence identity to the reference polynucleotide.

Nucleotide changes present in a variant polynucleotide may be silent, which means that these changes do not alter the amino acid sequences encoded by the reference polynucleotide.

Substitutions, additions and/or deletions can involve one or more nucleic acids. Alterations can produce conservative or non-conservative amino acid substitutions, deletions and/or additions.

Variants of a prey or a SID polypeptide encoded by a variant polynucleotide can possess a higher affinity of binding and/or a higher specificity of binding to its protein or polypeptide counterpart, against which it has been initially selected. In another context, variants can also loose their ability to bind to their protein or polypeptide counterpart.

By functional variant or fragment of a given polypeptide or polynucleotide is intended a variant or a fragment having the same function of the given polypeptide or polynucleotide.

The term “affinity of binding”, as used herein, can be defined as the affinity constant Ka when a given SID polypeptide of the present invention binds to a polypeptide and is the following mathematical relationship:

[SID/polypeptide complex]

Ka=______

[free SID] [free polypeptide]

wherein [free SID], [free polypeptide] and [SID/polypeptide complex] consist of the concentrations at equilibrium respectively of the free SID polypeptide, of the free polypeptide onto which the SID polypeptide binds and of the complex formed between SID polypeptide and the polypeptide onto which said SID polypeptide specifically binds.

The affinity of a SID polypeptide of the present invention or a variant thereof for its polypeptide counterpart can be assessed, for example, on a Biacore™ apparatus marketed by Amersham Pharmacia Biotech Company such as described by Szabo et al. (1995) and by Edwards and Leatherbarrow (1997).

As used herein the phrase “at least the same affinity” with respect to the binding affinity between a SID polypeptide of the present invention to another polypeptide means that the Ka is identical or can be at least two-fold, at least three-fold or at least five fold greater than the Ka value of reference.

As used herein, the term “modulating compound” means a compound that inhibits or stimulates or can act on another protein which can inhibit or stimulate the protein-protein interaction of a complex of two polypeptides or the protein-protein interaction of two polypeptides.

All Helicobacter pylori genes and proteins's names and sequences used herein are derived from the Tomb et al. (1997) publication on the H. pylori strain 26695 sequencing.

In the following description, the σ²⁸ factor and the anti-σ²⁸ factor will be designated by their ORF number, HP1032 and HP1122, respectively. SID1122 and SID1032 shall designate the Selected Interacting Domain (SID®) of HP1122 and HP1032, respectively, which are the specific domains of HP1122 and HP1032 involved in the interaction between HP1122 and HP1032.

Helicobacter pylori colonizes the human stomach and can cause gastroduodenal disease. Flagellar motility is an important factor of H. pylori for colonization of the gastric mucosa. The major flagellin subunit, FlaA, is transcriptionally controlled by the σ²⁸ factor of RNA polymerase encoded by fliA (HP1032).

In the present invention, the anti-σ²⁸ factor in H. pylori, called HP1122, using the Two-Hybrid screening System has been identified. The Two-Hybrid System consists in the construction of a library of random genomic fragments of the H. pylori strain 26695 and the screening of this library with a specific bait protein allowing the determination of preys which interact with this bait (see WO00/66277). Thus, HP1122, a new partner of an unknown function interacting with the σ²⁸ factor (HP1032) has been identified. It has been shown that (i) the C-terminal part of the HP1122 protein (residues 48-76) interacts with the σ²⁸ factor; and (ii) the region 4 of σ²⁸ interacts with HP1122 protein. In addition, HP1122 protein and SID1122 prevent association of σ²⁸ with the β subunit of RNA polymerase. This led to further investigations into whether HP1122 is the anti-σ²⁸ factor in H. pylori. This was confirmed using RNA slot blot hybridization, showing that (i) the σ²⁸-dependent transcription of the fleA promoter is increased in the HP1122-deleted strain and decreased after overexpression of HP1122. In addition, it was shown by electron microscopy that overexpression of HP1122 resulted in strongly truncated flagellar appendages. Thus, it was concluded that HP1122 is the anti-σ²⁸ factor, FlgM, in H. pylori.

This invention opens up new opportunities for the treatment of H. pylori infections, and more generally, to Gram negative and flagellated bacteria.

This invention provides a newly identified function of a Cj1464 protein and polynucleotide encoding the Cj1464 protein which is the anti-σ²⁸ factor of Campylobacter jejuni.

The Cj1464 amino acid sequence is: MINPIQQSYV ANTALNTNRI DKETKTNDTQ KTENDKASKI AEQIKNGTYK IDTKATAMI ADSLI (SEQ ID NO:9).

This invention provides a newly identified function of a PA3351 protein and polynucleotide encoding a PA3351 protein which is the anti-σ²⁸ factor of Pseudomonas aeruginosa.

The PA3351 amino acid sequence is: MVIDFNRLNP GSTPATTGRT GSTMGRPDA TGADKAGQM TSAPKSGESV QISETAQNMQ KVTDQLQTLP WDNDKVARI KQAIADGTYQ VDSERVASKL LDFESQR (SEQ ID NO:10).

This invention provides a newly identified function of HP1122 polypeptide and polynucleotide which is the anti-σ²⁸ factor of Helicobacter pylori.

This invention provides polypeptides SID1122 and SID1032 and polynucleotides encoding these polypeptides.

TABLE 1
SID1122, SID1032 and HP1122 nucleic acid and amino acid sequences

| SID1122 | SID1032 | HP1122 |
|---|---|---|
| Nucleic acid sequence: | Nucleic acid sequence: | Nucleic acid sequence: |
| atc aag aaa gcg att gaa aat | aaa gcg ctg aat caa atg agc | atg aat atc aaa tta aag gat ttt |
| aac cag tat aaa atc aac ttg | gaa aga gag caa atc ctt atc | aca atg att aat gcc gtt tct tct |
| cat gag act tct cac aaa atg | cag ctt tat tac ttt gaa gag ttg | ctt gct ccg gtg cag tct ttg ggg |
| gca aag gat tta ttg ggg ata | aat ttg agc gag att aaa gag | aat tat aag cgt gtg gaa aag |
| agc tag | att tta ggc att act gaa tcg cgc | aat gaa aaa gtt gaa aac aat |
| (SEQ ID NO:1) | att tct caa atc att aaa gaa | gag gcc gct ctt gat agg gta gct |
| | gtg att aaa aag gtg cgt aaa | gag atc aag aaa gcg att gaa |
| | tcc tta gga gtg gat cat ggc | aat aac cag tat aaa atc aac ttg |
| | tga | cat gag act tct cac aaa atg |
| | (SEQ ID NO:3) | gca aag gat tta ttg ggg ata agc |
| | | tag |
| | | (SEQ ID NO:5). |
| Amino acid sequence: | Amino acid sequence: | Amino acid sequence: |
| IKKAIENNQY KINLHETSHK | KALNQMSERE | MNIKLKDFTM INAVSSLAPV |
| MAKDLLGIS | QILIQLYYFE ELNLSEIKEI | QSLGNYKRVE |
| (SEQ ID NO:2) | LGITESRISQ IIKEVIKKVR | KNEKVENNEA ALDRVAEIKK |
| | KSLGVDHG | AIENNQYKIN LHETSHKMAK |
| | (SEQ ID NO:4) | DLLGIS |
| | | (SEQ ID NO:6). |

The present invention is not limited to the HP1122 as the anti-σ²⁸ factor or SID1122 and SID1032 nucleic acid and amino acid sequences as described in the Table 1, but also includes fragments of these sequences having at least 12 consecutive nucleic acids as well as variants thereof. The fragments or variants of these SID sequences possess at least the same affinity of binding to its protein or polypeptide counterpart, against which it has been initially selected. Moreover the variant and/or fragment of a given SID sequence can have between 95% and 99.99% sequence identity with the SID sequence, and variant and/or fragments of a given SID polynucleotide can have between 70% and 99.99% sequence identity with the SID polynucleotide.

According to the present invention the variants can be created by known mutagenesis techniques either in vitro or in vivo. Such a variant can be created so that it has altered binding characteristics with respect to the target protein and more specifically that the variant binds the target sequence with either higher or lower affinity.

Polynucleotides that are complementary to the above sequences SEQ ID NO:1, SEQ ID NO:3 and SEQ ID NO:5, their fragments, variants and those that have specific sequence identity are also included in the present invention.

Besides the isolated polynucleotides, SEQ ID NO:1, SEQ ID NO:3 and SEQ ID NO:5, encoding SID1122, SID1032 and HP1122 polypeptides, respectively, or fragments or variants thereof, can be inserted into a recombinant expression vector which contains the necessary elements for the transcription and translation of the inserted polypeptide-coding sequence. Such transcription elements include a regulatory region and a promoter. Thus, each polynucleotide of the present invention is operably linked to a promoter in the expression vector. The expression vector may also include a replication origin.

A wide variety of host/expression vector combinations are employed in expressing the polynucleotides of the present invention. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col EI, pCR1, pBR322, pMal-C2, pET, pGEX as described by Smith et al., pMB9 and derivatives thereof, plasmids such as RP4, phage DNAs such as the numerous derivatives of phage I such as NM989, as well as other phage DNA such as M13 and filamentous single stranded phage DNA; yeast plasmids such as the 2 micron plasmid or derivatives of the 2μ plasmid, as well as centromeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like.

For example, in a baculovirus expression system, both non-fusion transfer vectors, such as, but not limited to pVL941 (BamHI cloning site; Summers), pVL1393 (BamHI, SmaI, XbaI, EcoRI, NotI, XmaIII, BglII and PstI cloning sites; Invitrogen) pVL1392 (BglII, PstI, NotI, XmalII, EcoRI, XbalI, SmaI and BamHI cloning site; Summers and Invitrogen) and pBlueBacIII (BamHI, BglII, PstI, NcoI and HindIII cloning site, with blue/white recombinant screening, Invitrogen), and fusion transfer vectors such as, but not limited to, pAc700 (BamHI and KpnI cloning sites, in which the BamHI recognition site begins with the initiation codon; Summers), pAc701 and pAc70-2 (same as pAc700, with different reading frames), pAc360 (BamHI cloning site 36 base pairs downstream of a polyhedrin initiation codon; Invitrogen) and pBlueBacHisA, B, C (three different reading frames with BamHI, BglII, PstI, NcoI and HindIII cloning site, an N-terminal peptide for ProBond purification and blue/white recombinant screening of plaques; Invitrogen (220) can be used.

Mammalian expression vectors contemplated for use in the invention include vectors with inducible promoters, such as the dihydrofolate reductase promoters, any expression vector with a DHFR expression cassette or a DHFR/methotrexate co-amplification vector such as pED (PstI, SalI, Sbal, SmaI and EcoRI cloning sites, with the vector expressing both the cloned gene and DHFR; Kaufman, 1991). Alternatively a glutamine synthetase/methionine sulfoximine co-amplification vector, such as pEE14 (HindIII, XbalI, SmaI, SbaI, EcoRI and BclI cloning sites in which the vector expresses glutamine synthetase and the cloned gene; Ceiltech). A vector that directs episomal expression under the control of the Epstein Barr Virus (EBV) or nuclear antigen (EBNA) can be used such as pREP4 (BamHI, SfiI, XhoI, NotI, NheI, HindIII, NheI, PvuII and KpnI cloning sites, constitutive RSV-LTR promoter, hygromycin selectable marker; Invitrogen) pCEP4 (BamHI, SfiI, XhoI, NotI, NheI, HindIII, NheI, PvuII and KpnI cloning sites, constitutive hCMV immediate early gene promoter, hygromycin selectable marker; Invitrogen), pMEP4 (KpnI, PvuI, NheI, HindIII, NotI, XhoI, SfiI, BamHI cloning sites, inducible methallothionein IIa gene promoter, hygromycin selectable marker, Invitrogen), pREP8 (BamHI, XhoI, NotI, HindIII, NheI and KpnI cloning sites, RSV-LTR promoter, histidinol selectable marker; Invitrogen), pREP9 (KpnI, NheI, HindIII, NotI, XhoI, SfiI, BamHI cloning sites, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen).

Selectable mammalian expression vectors for use in the invention include, but are not limited to, pRc/CMV (HindIII, BstXI, NotI, SbaI and ApaI cloning sites, G418 selection, Invitrogen), pRc/RSV (HindIII, SpeI, BstXI, NotI, XbaI cloning sites, G418 selection, Invitrogen) and the like. Vaccinia virus mammalian expression vectors (see, for example Kaufman 1991) that can be used in the present invention include, but are not limited to, pSCI 1 (SmaI cloning site, TK- and β-gal selection), pMJ601 (SalI, SmaI, AflI, NarI, BspMII, BamHI, ApaI, NheI, SacII, KpnI and HindIII cloning sites; TK- and β-gal selection), pTKgptF1S (EcoRI, PstI, SalII, AccI, HindII, SbaI, BamHI and Hpa cloning sites, TK or XPRT selection) and the like.

Yeast expression systems that can also be used in the present invention include, but are not limited to, the non-fusion pYES2 vector (XbaI, SphI, ShoI, NotI, GstXI, EcoRI, BstXI, BamHI, Sacl, KpnI and HindIII cloning sites, Invitrogen), the fusion pYESHisA, B, C (XbalI, SphI, ShoI, NotI, BstXI, EcoRI, BamHI, SacI, KpnI and HindIII cloning sites, N-terminal peptide purified with ProBond resin and cleaved with enterokinase; Invitrogen), pRS vectors and the like.

Consequently, as part of the invention, mammalian and typically human cells, as well as bacterial, yeast, fungi, insect, nematode and plant cells that may be transformed by the one or several recombinant expression vectors comprising polynucleotides of the invention defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, Bacillus subtilis, Salmonella typhimurium, or strains of the genera of Pseudomonas, Streptomyces and Staphylococci.

Further suitable cells that can be used in the present invention include yeast cells such as those of Saccharomyces such as Saccharomyces cerevisiae.

The present invention also provides Helicobacter pylori recombinant host cell containing a pRH220catA comprising a polynucleotide of interest to overexpress the polypeptide encoded by the polynucleotide of interest. In another embodiment, the polynucleotide of interest is an Helicobacter pylori polynucleotide.

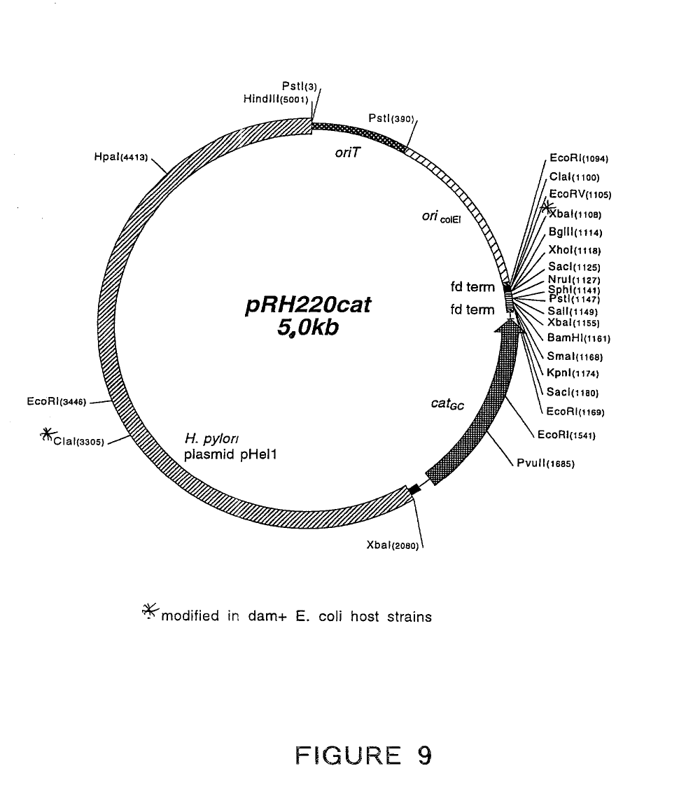

To obtain overexpression system in Helicobacter pylori recombinant host cell, the polynucleotide of interest is cloned in the multicopy plasmid pRH220cat (FIG. 9, Heuermann & Haas, 1998), under the control of the constitutive amiE promoter (Skouloubris et al., 1997). The amiE promoter fragment was obtained by PCR using the 1550-1551 primers (5′-CATGAGATCTCTATAAAAACAGAGCGGCTAAA-3′ (SEQ ID No. 11) and 5′-TGACGCATGCACTAGTCATATGATGTTCCTTGTTTTTTGATG-3′ (SEQ ID No. 12), respectively). The amplified fragment was cloned into the BglII-SphI sites of pRH220cat (FIG. 9) leading to plasmid pRH220catA. The plasmid pRH220catA containing the polynucleotide of interest is used to transform H. pylori.

Since HP1122 protein and HP1032 protein, HP1122 protein and SID1032, SID1122 and HP1032 protein and SID1122 and SID1032 interact, the present invention also provides complexes of two polypeptides: HP1122 protein (SEQ ID NO:6)-HP1032 protein (SEQ ID NO:8), HP1122 protein (SEQ ID NO:6)-SID1032 (SEQ ID NO:4), SID1122 (SEQ ID NO:2)-HP1032 protein (SEQ ID NO:8) and SID1122 (SEQ ID NO:2)-SID1032 (SEQ ID NO:4).

The HP1032 nucleic acid sequence is: atg att ttg atg atg gaa aat aga atg ccc aaa gga att caa aaa act gaa aca agc gaa aaa aat ata gaa aag gtt ttg aac gcc tat gat aag caa caa cac cac cat caa gac gat ctc gct att cag tat tta cca gcc gtg cgc gcc atg gcg ttt cgt cta aaa gag cgc ttg ccc agc tct att gat ttt aac gat ctg gtt tct att ggc act gaa gaa ttg att aaa tta gcc agg cgt tat gag agc gcg tta aac gat tct ttt tgg ggg tat gcg aag act cgt gtc aat ggg gcg atg tta gat tat ttg cgc tct tta gat gtg att tct cgc tct agc agg aaa ctc att aaa agc att gat att gaa atc acc aaa cac ctt aat gag cat ggg aaa gag cct agc gat gcg tat tta gcg caa act tta ggc gaa aat att gaa aaa att aaa gaa gcc aaa acg gct tca gat att tat gcg tta gtg cca ata gat gaa caa ttc aat gcg att gag caa gat gaa atc act aaa aaa att gaa gca gaa gag ttg tta gag cat gtc caa aaa gcg ctg aat caa atg agc gaa aga gag caa atc ctt atc cag ctt tat tac ttt gaa gag ttg aat ttg agc gag att aaa gag att tta ggc att act gaa tcg cgc att tct caa atc att aaa gaa gtg att aaa aag gtg cgt aaa tcc tta gga gtg gat cat ggc tga (SEQ ID NO:7).

The HP1032 amino acid sequence is: MILMMENRMP KGIQKTETSE KNIEKVLNAY DKQQHHHQDD LAIQYLPAVR AMAFRLKERL PSSIDFNDLV SIGTEELIKL ARRYESALND SFWGYAKTRV NGAMLDYLRS LDVISRSSRK LIKSIDIEIT KHLNEHGKEP SDAYLAQTLG ENIEKIKEAK TASDIYALVP IDEQFNAIEQ DEITKKIEAE ELLEHVQKAL NQMSEREQIL IQLYYFEELN LSEIKEILGI TESRISQIIK EVIKKVRKSL GVDHG (SEQ ID NO:8).

Also included as part of the invention are complexes of interacting fragments or variants of HP1122 protein-HP1032 protein, HP1122 protein-SID1032, SID1122-HP1032 protein and SID1122-SID1032.

In yet another embodiment, the present invention relates to an isolated complex of at least two polypeptides encoded by two polynucleotides wherein said two polypeptides are associated in the complex by affinity binding and are SEQ ID NO:2-SEQ ID NO:4, SEQ ID NO:2-SEQ ID NO:8, SEQ ID NO:4-SEQ ID NO:6, SEQ ID NO:6-SEQ ID NO:8.

The present invention is not limited to these polypeptide complexes alone but also includes the isolated complex of the two polypeptides in which fragments and/or homologous polypeptides exhibiting at least 80% sequence identity, as well as from 96% sequence identity to 99.99% sequence identity.

More specifically, as part of the invention, two interacting polypeptides comprising a polypeptide having:

at least 95% amino acid identity with SEQ ID NO:2 and a polypeptide having at least 95% amino acid identity with SEQ ID NO:4;

at least 95% amino acid identity with SEQ ID NO:2 and a polypeptide having at least 95% amino acid identity with SEQ ID NO:8;

at least 95% amino acid identity with SEQ ID NO:4 and a polypeptide having at least 95% amino acid identity with SEQ ID NO:6;

at least 95% amino acid identity with SEQ ID NO:6 and a polypeptide having at least 95% amino acid identity with SEQ ID NO:8.

Furthermore, the present invention provides a set of at least two polynucleotides comprising:

a first polynucleotide encoding SID1122 (SEQ ID NO:2) and a second polynucleotide encoding SID1032 (SEQ ID NO:4);

a first polynucleotide encoding SID1122 (SEQ ID NO:2) and a second polynucleotide encoding HP1032 (SEQ ID NO:8);

a first polynucleotide encoding SID1032 (SEQ ID NO:4) and a second polynucleotide encoding HP1122 (SEQ ID NO:6);

a first polynucleotide encoding HP1122 (SEQ ID NO:6) and a second polynucleotide encoding HP1032 (SEQ ID NO:8).

The invention also relates to a set of at least two polynucleotides of sequence:

SEQ ID NO:1 and SEQ ID NO:3;

SEQ ID NO:1 and SEQ ID NO:7;

SEQ ID NO:3 and SEQ ID NO:5;

SEQ ID NO:5 and SEQ ID NO:7;

and to a set of at least two polynucleotides comprising:

a first polynucleotide having at least 70% nucleic acid identity with sequence SEQ ID NO:1 and a second polynucleotide having at least 70% nucleic acid identity with sequence SEQ ID NO:3;

a first polynucleotide having at least 70% nucleic acid identity with sequence SEQ ID NO:1 and a second polynucleotide having at least 70% nucleic acid identity with sequence SEQ ID NO:7;

a first polynucleotide having at least 70% nucleic acid identity with sequence SEQ ID NO:3 and a second polynucleotide having at least 70% nucleic acid identity with sequence SEQ ID NO:5;

a first polynucleotide having at least 70% nucleic acid identity with sequence SEQ ID NO:5 and a second polynucleotide having at least 70% nucleic acid identity with sequence SEQ ID NO:7.

The set of at least two polynucleotides above described, can also be inserted into recombinant expression vectors which are described in detail above. Such a recombinant expression vector is part of the invention.

The present invention also relates to recombinant host cells transformed with the above-mentioned recombinant expression vectors. The recombinant host cells that can be used in the present invention were discussed in greater detail above.

A recombinant host cell of the present invention is, for example, a recombinant host cell expressing the two interacting polypeptides: HP1122 protein and HP1032 protein, HP1122 protein and SID1032, SID1122 and HP1032 protein and SID1122 and SID1032.

In yet another embodiment, the present invention relates to a method of selecting modulating compounds, as well as the modulating molecules or compounds themselves which may be used in a pharmaceutical composition.

The modulating compound can be selected according to a method which comprises:

cultivating on a selective medium a recombinant host cell with a modulating compound, said recombinant host cell expressing a first polypeptide comprising HP1122 or SID1122, or a fragment or a variant thereof, and a second polypeptide comprising HP1032 or SID1032, or a fragment or a variant thereof;

selecting said modulating compound which inhibits the interaction between the first and the second hybrid polypeptides and the growth of said recombinant host cell. In another embodiment of the method of selecting a modulating compound, the recombinant host cell is Helicobacter pylori.

The modulating compound can be selected according to a method which comprises:

cultivating on a selective medium a recombinant host cell with a modulating compound and a reporter gene the expression of which is toxic for said recombinant host cell wherein said recombinant host cell is transformed with two vectors:

wherein a first vector comprises a polynucleotide encoding a first hybrid polypeptide comprising HP1122 or SID1122, or a fragment or a variant thereof, fused to a DNA binding domain;

wherein a second vector comprises a polynucleotide encoding a second hybrid polypeptide comprising HP1032 or SID1032, or a fragment or a variant thereof, fused to a transcriptional activating domain that activates said toxic reporter gene when the first and second hybrid polypeptides interact;

selecting said modulating compound which inhibits the interaction between the first and the second hybrid polypeptides and allows the growth of said recombinant host cell. In another embodiment, the first hybrid polypeptide comprises HP1032 or SID1032, or fragment or variant thereof, fused to a DNA binding domain and the second hybrid polypeptide comprises HP1122 or SID1122, or fragment or variant thereof, fused to a transcriptional activating domain.

The modulating compound may also be selected according to a method which comprises:

cultivating on a selective medium a recombinant host cell with a modulating compound and a reporter gene the expression of which allows growth of the said recombinant host cell wherein said recombinant host cell is transformed with two vectors:

wherein a first vector comprises a polynucleotide encoding a first hybrid polypeptide comprising HP1122 or SID1122, or a fragment or a variant thereof, fused to a DNA binding domain;

wherein a second vector comprises a polynucleotide encoding a second hybrid polypeptide comprising HP1032 or SID1032, or a fragment or a variant thereof, fused to a transcriptional activating domain that activates said reporter gene when the first and second hybrid polypeptides interact;

selecting said modulating compound which inhibits or stimulates the growth of said recombinant host cell.

In another embodiment, the first hybrid polypeptide comprises HP1032 or SID1032, or fragment or variant thereof, fused to a DNA binding domain and the second hybrid polypeptide comprises HP1122 or SID1122, or fragment or variant thereof, fused to a transcriptional activating domain.

In another embodiment, the present invention relates to a method of selecting a modulating compound, which modulating compound inhibits or stimulates the interaction between HP1122 protein and HP1032 protein, HP1122 protein and SID1032, SID1122 and HP1032 protein and SID1122 and SID1032. This method comprises:

cultivating on a selective medium a recombinant host cell with a modulating compound and a reporter gene the expression of which is toxic for said recombinant host cell wherein said recombinant host cell is transformed with two vectors:

wherein a first vector comprises a polynucleotide encoding a first hybrid polypeptide comprising HP1122 or SID1122, or a fragment or a variant thereof, fused to a first domain of a protein;

wherein a second vector comprises a polynucleotide encoding a second hybrid polypeptide comprising HP1032 or SID1032, or a fragment or a variant thereof, fused to the second part of a complementary domain of the protein that activates said toxic reporter gene when the first and second hybrid polypeptides interact;

selecting said modulating compound which inhibits the interaction between the first and the second hybrid polypeptides and allows the growth of said recombinant host cell.

In another embodiment, the first hybrid polypeptide comprises HP1032 or SID1032, or fragment or variant thereof, fused to a first domain of a protein and the second hybrid polypeptide comprises HP1122 or SID1122, or fragment or variant thereof, fused to the second part of a complementary domain of the protein.

This method may also comprises:

cultivating on a selective medium a recombinant host cell with a modulating compound and a reporter gene the expression of which allows growth of the said recombinant host cell wherein said recombinant host cell is transformed with two vectors:

wherein a first vector comprises a polynucleotide encoding a first hybrid polypeptide comprising HP1122 or SID1122, or a fragment or a variant thereof, fused to a first domain of a protein;

wherein a second vector comprises a polynucleotide encoding a second hybrid polypeptide comprising HP1032 or SID1032, or a fragment or a variant thereof, fused to the second part of a complementary domain of the protein that activates said reporter gene when the first and second hybrid polypeptides interact;

selecting said modulating compound which inhibits or stimulates the growth of said recombinant host cell.

In another embodiment, the first hybrid polypeptide comprises HP1032 or SID1032, or fragment or variant thereof, fused to a first domain of a protein and the second hybrid polypeptide comprises HP1122 or SID1122, or fragment or variant thereof, fused to the second part of a complementary domain of the protein.

In another embodiment, the method comprises the steps of:

cultivating on a selective medium a recombinant host cell with a modulating compound and a reporter gene the expression of which is toxic for said recombinant host cell wherein said recombinant host cell is transformed with two vectors:

wherein a first vector comprises a polynucleotide encoding a first hybrid polypeptide comprising HP1122 or SID1122, or a fragment or a variant thereof, fused to a first domain of an enzyme;

wherein a second vector comprises a polynucleotide encoding a second hybrid polypeptide comprising HP1032 or SID1032, or a fragment or a variant thereof, fused to an enzymatic transcriptional activating domain that activates said toxic reporter gene when the first and second hybrid polypeptides interact;

selecting said modulating compound which inhibits the interaction between the first and the second hybrid polypeptides and allows the growth of said recombinant host cell.

In another embodiment, the first hybrid polypeptide comprises HP1032 or SID1032, or fragment or variant thereof, fused to a first domain of an enzyme and the second hybrid polypeptide comprises HP1122 or SID1122, or fragment or variant thereof, fused to the second part of a complementary domain of the enzyme.

In another embodiment, the method comprises the steps of:

cultivating on a selective medium a recombinant host cell with a modulating compound and a reporter gene the expression of which allows growth of the said recombinant host cell wherein said recombinant host cell is transformed with two vectors:

wherein a first vector comprises a polynucleotide encoding a first hybrid polypeptide comprising HP1122 or SID1122, or a fragment or a variant thereof, fused to a first domain of an enzyme;

wherein a second vector comprises a polynucleotide encoding a second hybrid polypeptide comprising HP1032 or SID1032, or a fragment or a variant thereof, fused to an enzymatic transcriptional activating domain that activates said toxic reporter gene when the first and second hybrid polypeptides interact;

selecting said modulating compound which inhibits or stimulates the growth of said recombinant host cell.

In another embodiment, the first hybrid polypeptide comprises HP1032 or SID1032, or fragment or variant thereof, fused to a first domain of an enzyme and the second hybrid polypeptide comprises HP1122 or SID1122, or fragment or variant thereof, fused to the second part of a complementary domain of the enzyme.

In the three methods described above any toxic reporter gene can be utilized including those reporter genes that can be used for negative selection including the URA3 gene, the CYH1 gene, the CYH2 gene and the like.

In the selection methods described above, the activating domain can be p42 Gal 4, YP16 (HSV) and the DNA-binding domain can be derived from Gal4 or Lex A. The protein or enzyme can be adenylate cyclase, guanylate cyclase, DHFR and the like.

In yet another embodiment, the present invention provides a kit for screening a modulating compound. This kit comprises a recombinant host cell which comprises a reporter gene. The host cell is transformed with two vectors. The first vector comprises a polynucleotide encoding a first hybrid polypeptide comprising HP1122 or SID1122, or fragment or variant thereof, fused to a DNA binding domain; and a second vector comprises a polynucleotide encoding a second hybrid polypeptide comprising HP1032 or SID1032, or fragment or variant thereof, fused to a transcriptional activating domain that activates said reporter gene when the first and second hybrid polypeptides interact.

In another embodiment, the expression of the said reporter gene is toxic for the recombinant host cell.

In yet another embodiment a kit is provided for screening a modulating compound by providing a recombinant host cell, as described in the paragraph above, but instead of a DNA binding domain, the first vector comprises a first hybrid polypeptide containing a first domain of a protein and instead of the transcriptional activating domain, the second vector comprises a second polypeptide containing the second part of a complementary domain of the protein that activates the reporter gene when the first and second hybrid polypeptides interact.

In another embodiment, the expression of the said reporter gene is toxic for the recombinant host cell.

Thus, the present invention relates to modulating compounds obtained with the previously described methods. These modulating compounds may act as a cofactor, as an inhibitor, as antibodies, as tags, as a competitive inhibitor, as an activator or alternatively have agonistic or antagonistic activity on the protein-protein interactions.

The activity of the modulating compound does not necessarily, for example, have to be 100% activation or inhibition. Indeed, even partial activation or inhibition can be achieved that is of pharmaceutical interest.

Such compounds can be used in a pharmaceutical composition to treat or prevent Gram negative flagellated bacteria infection, more specifically Helicobacter sp. or Campylobacter jejuni or Pseudomonas aeruginosa infection, in particular Helicobacter pylori infection, in a human or mammal.

The present invention relates to a modulating compound that inhibits the interaction between protein encoded by HP1032 and any protein interacting with protein encoded by HP1032, such as β subunit of Helicobacter pylori RNA polymerase.

The present invention also relates to a modulating compound that activates the interaction between HP1122 protein and HP1032 protein, HP1122 protein and SID1032, SID1122 and HP1032 protein and SID1122 and SID1032.

It has been demonstrated that HP1122 protein (SEQ ID NO:6) and SID1122 (SEQ ID NO:2) exhibit an anti-σ²⁸ factor activity in Helicobacter pylori and are used as modulating compounds, an object of the present invention.

In yet another embodiment, the present invention relates to a pharmaceutical composition comprising the modulating compound of the invention for preventing or treating Gram negative flagellated bacteria infection, such as Helicobacter sp. or Campylobacter jejuni or Pseudomonas aeruginosa infection, or Helicobacter pylori infection in a human or a mammal. Such pharmaceutical composition may also be used for preventing or treating gastric ulcers and gastric cancers in a human or animal, most preferably in a mammal.

This pharmaceutical composition comprises a pharmaceutically acceptable amount of the modulating compound. The pharmaceutically acceptable amount can be estimated from cell culture assays. For example, a dose can be formulated in animal models to achieve a circulating concentration range that includes or encompasses a concentration point or range having the desired effect in an in vitro system. This information can thus be used to accurately determine the doses in other mammals, including humans and animals.

The therapeutically effective dose refers to that amount of the compound that results in amelioration of symptoms in a patient. Toxicity and therapeutic efficacy of such compounds can be determined by standard pharmaceutical procedures in cell cultures or in experimental animals. For example, the LD50 (the dose lethal to 50% of the population) as well as the ED50 (the dose therapeutically effective in 50% of the population) can be determined using methods known in the art. The dose ratio between toxic and therapeutic effects is the therapeutic index which can be expressed as the ratio between LD50 and ED50 compounds that exhibit high therapeutic indexes.

The data obtained from the cell culture and animal studies can be used in formulating a range of dosage of such compounds which lies preferably within a range of circulating concentrations that include the ED50 with little or no toxicity.

The pharmaceutical composition can be administered via any route such as locally, orally, systemically, intravenously, intramuscularly, mucosally, using a patch and can be encapsulated in liposomes, microparticles, microcapsules, and the like. The pharmaceutical composition can be embedded in liposomes or even encapsulated.

Any pharmaceutically acceptable carrier or adjuvant can be used in the pharmaceutical composition. The modulating compound will be preferably in a soluble form combined with a pharmaceutically acceptable carrier. The techniques for formulating and administering these compounds can be found in “Remington's Pharmaceutical Sciences” Mack Publication Co., Easton, Pa., latest edition.

The mode of administration, optimum dosages and galenic forms can be determined by the criteria known in the art taken into account the seriousness of the general condition of the mammal, the tolerance of the treatment and the side effects.

The pharmaceutical composition comprises a modulating compound identified by one of the methods previously described.

In yet another embodiment, the present invention relates to a pharmaceutical composition comprising HP1122 protein (SEQ ID NO:6) or SID1122 polypeptide (SEQ ID NO:2) or Cj1464 protein (SEQ ID NO:9) or PA3351 (SEQ ID NO:10), or fragment or variant thereof. The HP1122 protein or SID1122 polypeptide, fragment or variant thereof can be used in a pharmaceutical composition provided that it is endowed with specific binding properties to HP1032 protein.

The original properties of the SID1122 polypeptide or variants thereof interfere with the naturally occurring interaction between HP1122 protein and HP1032 protein within Helicobacter pylori.

Thus, the present invention relates to a pharmaceutical composition comprising a pharmaceutically acceptable amount of a HP1122 protein or SID1122 polypeptide, or fragment or variant thereof, provided that the fragment or the variant has the two following characteristics; i.e., that it is endowed with highly specific binding properties to HP1032 protein and is devoid of biological activity of the natural HP1032 protein.

The Cj1464 protein, HP1122 protein or SID1122 polypeptide as active ingredients will be preferably in a soluble form combined with a pharmaceutically acceptable carrier. The techniques for formulating and administering these compounds can be found in “Remington's Pharmaceutical Sciences” supra.

The amount of pharmaceutically acceptable Cj1464, PA3351, HP1122 protein or SID1122 polypeptide can be determined as described above for the modulating compounds using cell culture and animal models.

In another embodiment, the present invention relates to a pharmaceutical composition comprising a pharmaceutically effective amount of SEQ ID NO:1 or SEQ ID NO:5 polynucleotide encoding SID1122 polypeptide or HP1122 protein, respectively, or a fragment or a variant thereof wherein the SEQ ID NO:1 or SEQ ID NO:5 polynucleotide is placed under the control of an appropriate regulatory sequence. Appropriate regulatory sequences that are used are polynucleotide sequences derived from promoter elements and the like.

The pharmaceutical composition of the present invention can also comprise the polynucleotides of sequence SEQ ID NO:1 or SEQ ID NO:5 which code for the polypeptide of sequence SEQ ID NO:2 or SEQ ID NO:5 and/or functional variants thereof, wherein the pharmaceutical compound is administered to modulate complexion of polypeptides comprising at least HP1032 by way of gene therapy. Any of the methodologies relating to gene therapy available within the art may be used in the practice of the present invention such as those described by Goldspiel et al. (1993).

Besides the HP1122 and SID1122 polynucleotide, the pharmaceutical composition of the present invention can include a recombinant expression vector comprising the SEQ ID NO:1 or SEQ ID NO:5 polynucleotide encoding the SID1122 polypeptide or the HP1122 protein, respectively, fragment or variant thereof.

Delivery of the therapeutic polynucleotide into a patient may be direct in vivo gene therapy (i.e., the patient is directly exposed to the nucleic acid or nucleic acid-containing vector) or indirect ex vivo gene therapy (i.e., cells are first transformed with the nucleic acid in vitro and then transplanted into the patient).

For example, for in vivo gene therapy, an expression vector containing the polynucleotide is administered in such a manner that it becomes intracellular; i.e., by infection using a defective or attenuated retroviral or other viral vectors as described, for example in U.S. Pat. No. 4,980,286 or by Robbins et al. (1998).

The various retroviral vectors that are known in the art are such as those described in Miller et al. (1993) which have been modified to delete those retroviral sequences which are not required for packaging of the viral genome and subsequent integration into host cell DNA. Also adenoviral vectors can be used which are advantageous due to their ability to infect non-dividing cells and such high-capacity adenoviral vectors are described in Kochanek (1999). Chimeric viral vectors that can be used are those described by Reynolds et al. (1999). Hybrid vectors can also be used and are described by Jacoby et al. (1997).

The compositions comprising the expression vectors can contain physiological acceptable carriers such as diluents, adjuvants, excipients and any vehicle in which this composition can be delivered therapeutically and can include, but are not limited to sterile liquids such as water and oils.

Direct injection of naked DNA or through the use of microparticle bombardment (e.g., Gene Gun®; Biolistic, Dupont) or by coating it with lipids can also be used in gene therapy. Cell-surface receptors/transfecting agents or through encapsulation in liposomes, microparticles or microcapsules or by administering the nucleic acid in linkage to a peptide which is known to enter the nucleus or by administering it in linkage to a ligand predisposed to receptor-mediated endocytosis (See, Wu & Wu, 1987) can be used to target cell types which specifically express the receptors of interest.

In ex vivo gene therapy, a gene is transferred into cells in vitro using tissue culture and the cells are delivered to the patient by various methods such as injecting subcutaneously, application of the cells into a skin graft and the intravenous injection of recombinant blood cells such as hematopoietic stem or progenitor cells.

Cells into which a polynucleotide can be introduced for the purposes of gene therapy include, for example, epithelial cells, endothelial cells, keratinocytes, fibroblasts, muscle cells, hepatocytes and blood cells. The blood cells that can be used include, for example, T-lymphocytes, B-lymphocytes, monocytes, macrophages, neutrophils, eosinophils, megakaryocytes, granulocytes, hematopoietic cells or progenitor cells and the like.

In another embodiment a polynucleotide ligand compound may be produced in which the ligand comprises a fusogenic viral peptide designed so as to disrupt endosomes, thus allowing the polynucleotide to avoid subsequent lysosomal degradation. The polynucleotide may be targeted in vivo for cell specific endocytosis and expression by targeting a specific receptor such as that described in WO92/06180, WO93/14188 and WO93/20221. Alternatively the polynucleotide may be introduced intracellularly and incorporated within the host cell genome for expression by homologous recombination (See Zijlstra et al., 1989).

The pharmaceutical composition of the present invention can also include a recombinant host cell containing a recombinant expression vector comprising the SEQ ID NO:1 or SEQ ID NO:5 polynucleotide encoding the SID1122 polypeptide or HP1122 protein, respectively, a functional fragment or variant thereof.

The above described pharmaceutical compositions can be administered by any route such as orally, systemically, intravenously, intramuscularly, intradermally, mucosally, encapsulated, using a patch and the like. Any pharmaceutically acceptable carrier or adjuvant can be used in this pharmaceutical composition.

The present invention relates to a therapeutic composition comprising an antibacterial substrate capable of modulating the activity of HP1032.

The therapeutic composition of the invention may also comprise an active molecule capable of interacting with HP1032 to produce a product toxic for Helicobacter pylori.

In yet another embodiment the present invention relates to the use of Cj1464, PA3351, HP1122, HP1032, SID1122 and/or SID1032 in protein chips or protein microarrays. It is well known in the art that microarrays can contain more than 10,000 spots of a protein that can be robotically deposited on a surface of a glass slide or nylon filter. The proteins covalently attached to the slide surface, still retain their ability to interact with other proteins or small molecules in solution. In some instances the protein samples can be made to adhere to glass slides by coating the slides with an aldehyde-containing reagent that attaches to primary amines. A process for creating microarrays is described, for example by MacBeath and Schreiber (2000) or Service, Science, Vol, 289, Number 5485 pg. 1673 (2000). An apparatus for controlling, dispensing and measuring small quantities of fluid is described, for example, in U.S. Pat. No. 6,112,605.

The present invention provides antibodies directed against Cj1464, PA3351, HP1122, HP1032, SID1122, SID1032, or fragments or variants thereof.

For example, a method to obtain such antibodies may be the following. The polypeptide of interest is injected into mice and polyclonal and monoclonal antibodies are made following the procedure set forth in Sambrook et al (1989).

More specifically, mice are immunized with an immunogen comprising Cj1464, PA3351, HP1122, HP1032, SID1122 or SID1032, or fragment or variant thereof, conjugated to keyhole limpet hemocyanin using glutaraldehyde or EDC as is well known in the art. The complexes can also be stabilized by crosslinking as described in WO00/37483. The immunogen is then mixed with an adjuvant. Each mouse receives four injections of 10 μg to 100 ug of immunogen, and after the fourth injection, blood samples are taken from the mice to determine if the serum contains antibodies to the immunogen. Serum titer is determined by ELISA or RIA. Mice with sera indicating the presence of antibody to the immunogen are selected for hybridoma production.

Spleens are removed from immune mice and single-cell suspension is prepared (Harlow et al 1988). Cell fusions are performed essentially as described by Kohler et al. Briefly, P365.3 myeloma cells (ATTC Rockville, Md.) or NS-1 myeloma cells are fused with spleen cells using polyethylene glycol as described by Harlow et aL (1989). Cells are plated at a density of 2×10⁵ cells/well in 96-well tissue culture plates. Individual wells are examined for growth and the supernatants of wells with growth are tested for the presence of the “polypeptide of interest”-specific antibodies by ELISA or RIA using the polypeptide of interest, as a target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to either Cj1464, PA3351, HP1122, HP1032, SID1122 or SID1032, or fragments or variants thereof, to check whether they are specific to each of these polypeptides.

Monoclonal antibodies against each of the SID1122 or SID1032, or fragments or variants thereof, are prepared in a similar manner by mixing these polypeptides together, immunizing an animal, fusing spleen cells with myeloma cells and isolating clones which produce antibodies specific for the protein complex, but not for individual proteins.

The present invention also provides a process of preparation of Cj1464, PA3351, HP1122, SID1122 or SID1032 polypeptides, or fragments or variants thereof, comprising the steps of:

culturing under suitable conditions a prokaryotic or eukaryotic host cell transformed or transfected with a polynucleotide encoding either Cj1464, PA3351, HP1132, SID1122 or SID1032 polypeptides, or fragments or variants thereof, in a manner allowing the host cell to express said polypeptide; and

optionally isolating the desired polypeptide expression product.

The present invention also relates to a method of treating or preventing Gram negative flagellated bacteria infection in a human or mammal, more specifically Helicobacter sp. or Campylobacter jejuni or Pseudomonas aeruginosa infection, in particular Helicobacter pylori infection.

This method comprises administering to a human or a mammal in need of such treatment a pharmaceutically effective amount of a pharmaceutical composition described above. In another embodiment, the modulating compound is a polynucleotide which may be placed under the control of a regulatory sequence which is functional in the mammal or human.

In this method, the pharmaceutical composition may be administered by any route such as oral route, intradermal route, inramuscular route, intravenous route or mucosal route.

Thus, the present invention also relates to a method of preventing or treating Gram negative flagellated bacteria infection in a human or mammal, such as Helicobacter sp. or Campylobacter jejuni or Pseudomonas aeruginosa infection, or Helicobacter pylori infection in a human or a mammal said method comprising the steps of administering to a human or a mammal in need of such treatment a pharmaceutically effective amount of:

a SID1122 polypeptide of SEQ ID NO:2, HP1122 polypeptide of SEQ ID NO:6, Cj1464 of SEQ ID NO:9, or PA3351 of SEQ ID NO:10 or a fragment or a variant thereof; or

a SID1122 polynucleotide of SEQ ID NO:1 encoding a SID1122 polypeptide, or HP1122 polynucleotide of SEQ ID NO:5 encoding HP1122 protein or a variant or a fragment thereof wherein said polynucleotide is placed under the control of a regulatory sequence which is functional in said mammal; or

a recombinant expression vector comprising a polynucleotide encoding the SID1122 polypeptide or the HP1122 protein or the Cj1464 protein or the PA3351 protein or fragment or variant thereof.

In order to fully illustrate the present invention and advantages thereof, the following specific examples are given, it being understood that the same are intended only as illustrative and in nowise limitative.

### EXAMPLES

Bacterial Strains, Plasmids and Medium

Medium compositions and standard protocols are available in Sambrook and Maniatis (1989). DO-2 and DO-3 correspond to “Drop Out”-Leu-Trp medium and “Drop Out”-Leu-Trp-His medium, respectively.

Both isogenic mutants fliA and HP1122 of H. pylori N6 were obtained by allelic exchange mutagenesis. The pILL570-1 vector was constructed by restricting pILL570 (Labigne et al., 1992) with ClaI-HindIII and ligating the following linker: 5′-ATCGATGCGGCCGCGAATTCAAGCTT-3′ (SEQ ID No. 13). In one plasmid, pILL570-1-1032K, the fliA gene was inactivated by the insertion of a nonpolar cassette (subcloned from pUC18K2 (Ménard et al., 1993)) composed of the aphA-3 kanamycin resistance gene with its promoter and terminator regions deleted but with an additional ribosome site (RBS) downstream of the aphA-3 gene. This kanamycin cassette was obtained by polymerase chain reaction (PCR) with primer pairs 2386-2387 (5′-GCTCGGTACCCGGGTGACTAAC-3′ SEQ ID No. 14) and 5′-CTTCCCCCGGGCATTATTCCCTCCAGG-3′(SEQ ID No. 15), respectively) and inserted in the unique SspI site of the fliA gene at position 454. The second plasmid, pILL570-1-1122K, carried the aphA-3 kanamycin resistance gene (from pILL600, from Labigne-Roussel et al., 1988), encompassed by both fragments (521 bp each) obtained by PCR performed on H. pylori 26665 chromosomal DNA with primer pairs 2388-2389 (5′-CCATCGATCTCACACGCTTAGACGCTM-3′ (SEQ ID No. 16) and 5′-GGACTAGTCTAAGTTAAAAGCCTTAAGAT-3′ (SEQ ID No. 17), respectively) and 2391-2392 (5′-CGCGGATCCTTTTAAGAAAGGTGTTT-3′ (SEQ ID No. 18) and 5′-TTTTCTGCAGGCCMCGCCCTTTTGGT-3′ (SEQ ID No. 19), respectively). The kanamycin cassette was inserted at position 119 of the HP1122 coding sequence. H. pylori fliA and HP1122 mutants were produced by allelic exchange following transformation with 2 μg of pILL570-1-1032K and pILL570-1-1122K of H. pylori N6, respectively. Bacteria showing chromosomal allelic exchange with pILL570-1-1032K or pILL570-1-1122K were selected on kanamycin (20 μg/ml) and confirmed by PCR with the appropriate oligonucleotides.

To obtain overexpression of HP1122, the HP1122 gene was cloned in the multicopy plasmid pRH220cat (FIG. 9, Heuermann & Haas, 1998), under the control of the constitutive amiE promoter (Skouloubris et al., 1997). The amiE promoter fragment was obtained by PCR using the 1550-1551 primers (5′-CATGAGATCTCTATAAAAACAGAGCGGCTAAA-3′ (SEQ ID No. 11), and 5′-TGACGCATGCACTAGTCATATGATGTTCCTTGTITTTTGATG-3′ (SEQ ID No. 12), respectively). The amplified fragment was cloned into the BglII-SphI sites of pRH220cat (FIG. 9) leading to plasmid pRH220catA. The HP1122 coding sequence was obtained by PCR using the 1777-1669 primers (5′-GGGAATTCCATATGAATATCAAATTAAAGGAT-3′ (SEQ ID No. 20), and 5′-ATCGCGGATCCCTAGCTTATCCCCAATAAATCCTT-3′ (SEQ ID No. 21), respectively) and was cloned into the NdeI-BamHI sites of pRH220catA located just downstream of the amiE promoter giving plasmid pRH220catA-HP1122. 2 μg of pRH220catA-HP1122 were then used to transform H. pylori strain N6. Colonies containing the pRH220catA-HP1122 vector were selected on chloramphenicol (4 μg/ml) containing medium and confirmed by PCR.

All PCR fragments were checked by sequencing and mutagenesis was confirmed by PCR. H. pylori strains were cultured on blood agar-base 2 plates supplemented with 10% horse blood and the following antibiotics: vancomycin (10 mg/liter), polymyxin B (2,500 U/liter), trimethoprim (5 mg/liter) and amphotericin B (4 mg/liter). Plates were incubated at 37° C. under microaerobic conditions.

### Example 1

### HP1122 Interacts with Region 4 of the σ^{∞}Factor of RNA Polymerase

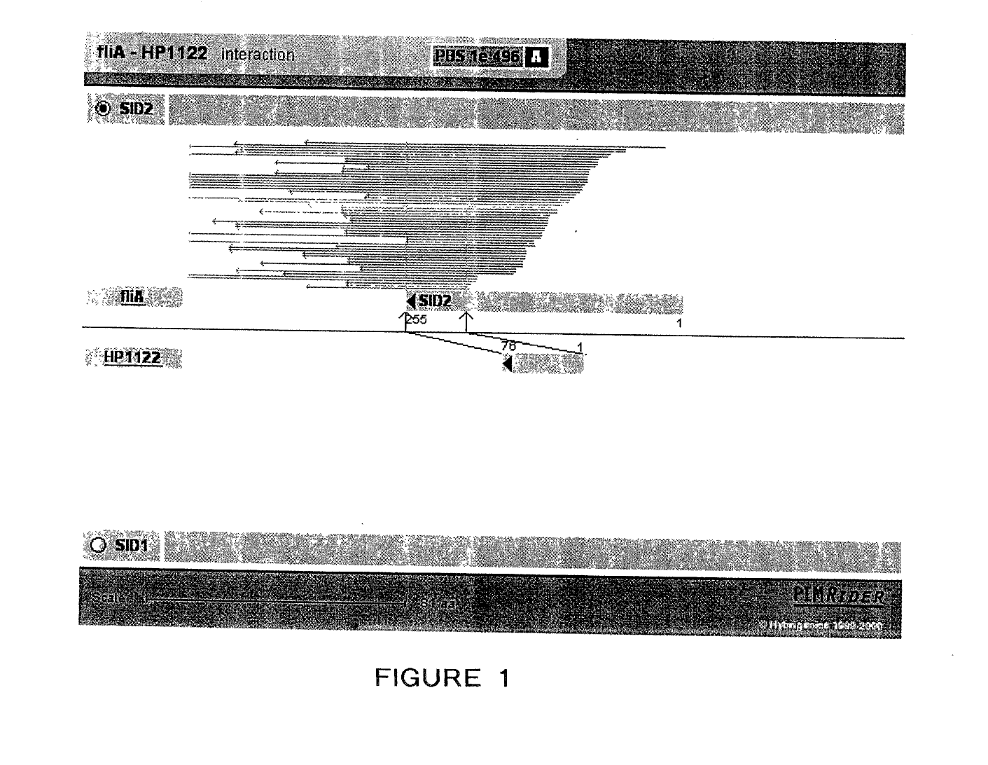

Based on the availability of the sequence of the H. pylori strain 26695 (Tomb et al., 1997), a large scale protein-protein interaction map was previously established (Rain et al., 2001). The σ²⁸ protein interaction map showed connections between σ²⁸ and ββ′ fused subunits, which are encoded by the rpoBC gene. In addition, it was found that σ²⁸ protein interacts significantly with HP1122 protein, a protein of unknown function. In this context, 60 independent fragments of HP1122 were identified as interacting with the σ²⁸ protein (Rain et al., 2001). To confirm this interaction, HP1122 was used as a bait for screening the complex library of prey polypeptides. In this experiment, 36 independent fragments of σ²⁸ were identified as contacting the HP1122 protein (FIG. 1). Both screenings strongly suggested that HP1122 protein interacts with the σ²⁸ factor of RNA polymerase.

These interactions were identified with the Mating Two Hybrid System as described in the WO00/66722 patent application.

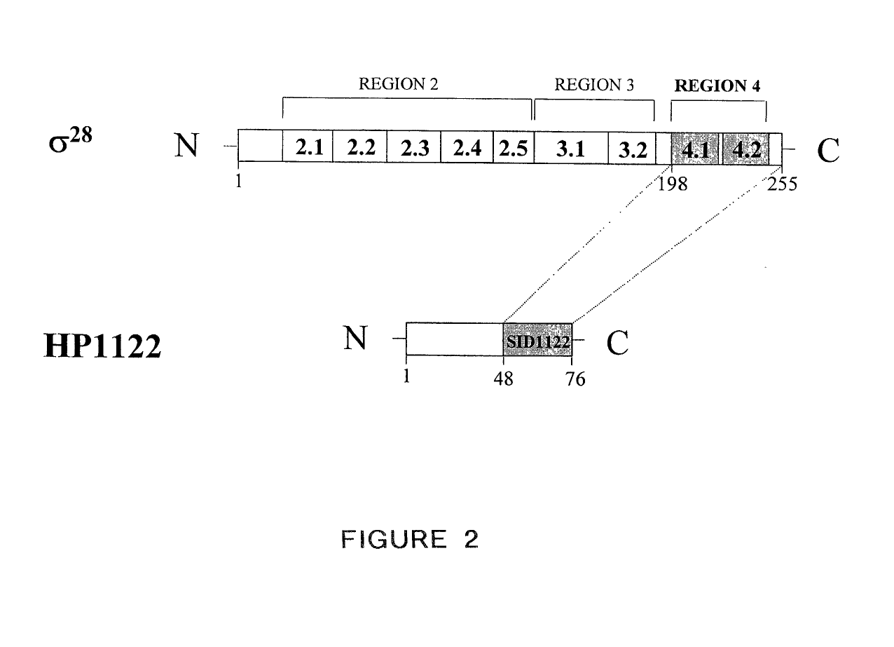

The common sequence shared by the independent fragments is referred to as the Selected Interacting Domain (SID®). The SID of σ²⁸, was identified as corresponding to residues 198-255 (SID1032), is located in region 4 of this protein and that the SID® of HP1122 (SID1122: residues 48-76) is located at the C-terminal part (FIG. 2). All these data suggest that the C-terminal part of HP1122 protein interacts with region 4 of σ²⁸. This mode of interaction, associated with the small size of HP1122 (8 kDa) led to the conclusion that HP1122 was be the anti-σ²⁸ factor, FlgM, of H. pylori.

### Example 2

### HP1122 Protein Inhibits Interaction Between σ²⁸ and β Subunits of RNA Polymerase

The primary function of an anti-σ²⁸ factor is to inhibit RNA polymerase activity, either by blocking the interaction between the σ factor and the core enzyme, or by counteracting promoter recognition by the σ factor (for review, Hughes K T & Matthai, 1998). Concerning the anti-σ factor FlgM, Ohnishi et al. (1992) have shown that binding of FlgM to σ²⁸ prevents its association with the RNA polymerase core enzyme. To confirm that HP1122 protein exhibits an anti-σactivity, the three-hybrid system was used which allowed to test the effect of a third partner, the HP1122 protein, on a two-hybrid interaction, σ²⁸ and ββ′ (Tirode et al., 1997).

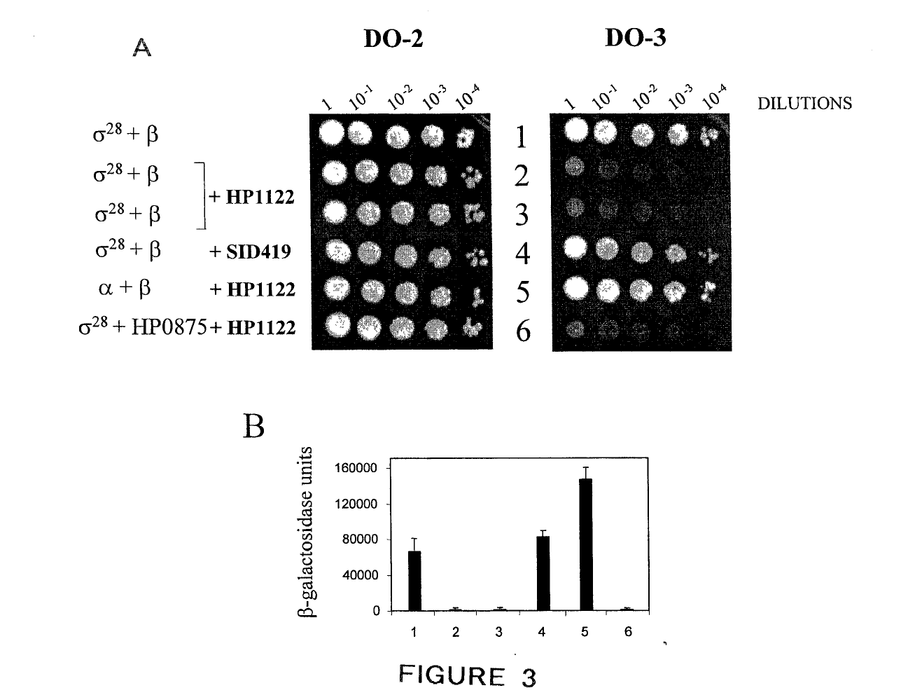

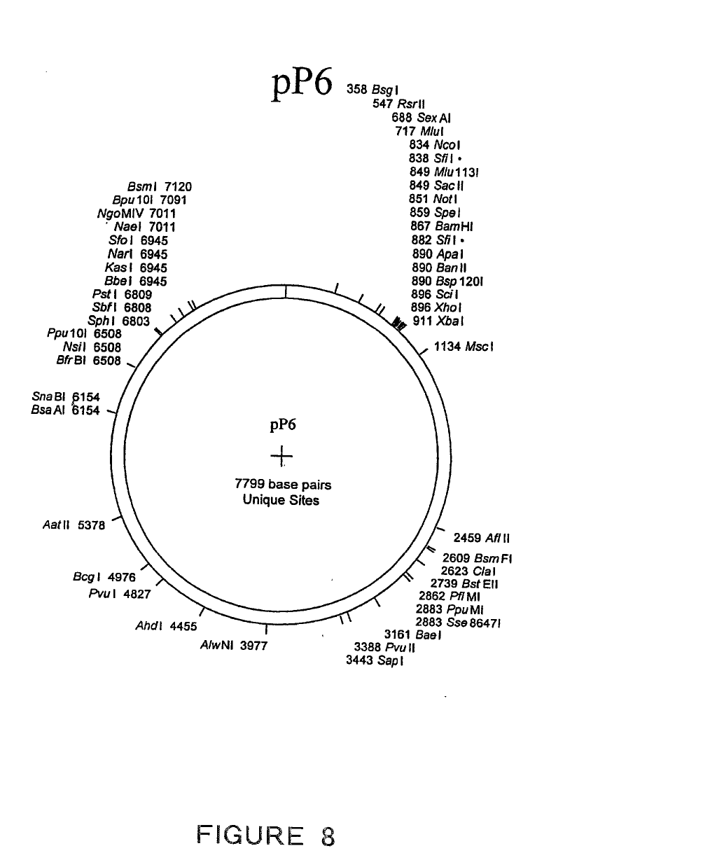

In the three-hybrid system, a fragment of the β protein (nucleotides 1674-4061) was expressed fused to the GAL4 Activation Domain (AD) in the pP6 plasmid (FIG. 8), whereas σ²⁸ was introduced in the p3H1 vector in fusion with the DNA-binding domain (DBD) of GAL4. In addition, this vector contains the Met25 promoter which allows expression of a third partner in medium lacking methionine. After transformation of Y187 and CG1945 yeast cells by the pP6(β) and p3H1-σ²⁸ vectors, respectively, both strains were mated. The resulting diploid strain was grown on a minimal medium lacking leucine and tryptophan to select for both plasmids (DO-2) and on DO-2 without histidine to select for interaction (DO-3). As a positive control, this strain was observed to grow on the selective medium for dilutions ranging from 1 to 10⁻⁴ and to give a strong β-galactosidase activity (FIGS. 3A and B, lane 1). This result shows an interaction between σ²⁸ and β proteins, as previously identified using library screening (Rain et al., 2001).

Two different plasmids were used for this study: (i) the pP6 vector (FIG. 8) which contain the GAL4 activation domain (AD) (Rain et al., 2001). One of the β fragments (nucleotides 1674-4061) obtained by screening the σ²⁸ protein was selected and used as prey in the pP6 vector fused to GAL4 AD; (ii) the p3H1 vector which contains the DNA-binding domain (DBD) of GAL4 and a methionine-regulated Met25 promoter (Tirode et al., 1997). The σ²⁸ encoding sequence of 765 bp was cloned into the BamHI/PstI sites of p3H1 as fusion protein with GAL4-DBD giving p3H1-σ²⁸. In addition, the HP1122 coding sequence was amplified by PCR using the 1783-1784 primers (5′-ATTTGCGGCCGCAAATATCAAATTAAAGGATTTT-3′ (SEQ ID No. 22), and 5′-GGACTAGATCTGCTTATCCCCMTAAATCCTT-3′ (SEQ ID No. 23), respectively) and was cloned into the NotI/BglII sites of p3H1-σ²⁸ under the control of the Met25 promoter to result in the p3H1-σ²⁸-HP1122 recombinant plasmid. Expression from the Met25 promoter is obtained in the absence of methionine. As negative controls, (i) a prey corresponding to a fragment of the HP0875 gene (nucleotides from 127 to 1518) has been used; (ii) a HP0419 fragment (nucleotide from 333 to 783) obtained by PCR with the 1585-1586 primers (5′-ATTTGCGGCCGCATCTTTGGGGGTAGAGGATTTGCAT-3′ (SEQ ID No. 24), and 5′-GGACTAGATCTACGCTTGCTTGGTTTAAGCATTTT-3′, (SEQ ID No. 25), respectively) was cloned in the NotI/BglII sites of p3H1-σ²⁸; (iii) and the HP1293 gene encoding the a subunit of RNA polymerase was cloned in the p3H1 vector. All PCR fragments and in frame fusions were checked by sequencing.

The pP6 (FIG. 8) and p3HI derived-plasmids were used to transform the Y187 and CG1945 yeast strains, respectively. Both strains were mated in YPD buffer (Yeast Peptone Dextrose; Bio 101, Inc) for 4 hours at 30° C. and the resulting diploid strain was selected on a minimal medium lacking leucine and tryptophane (DO-2). The interaction between proteins was observed in plates containing DO-2 deleted in histidine (DO-3) without methionine. To quantify this effect, LacZ activity was measured in a luminometric assay (Tropix).

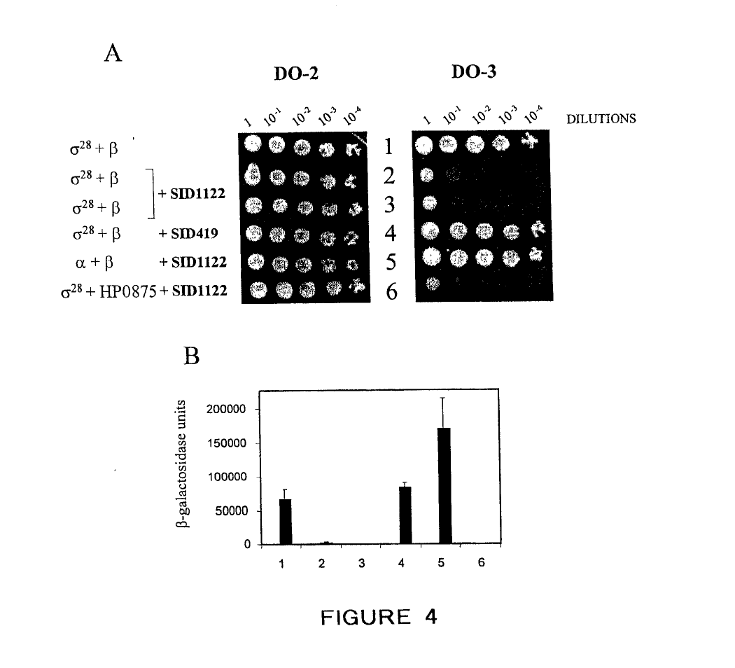

To assay whether HP1122 protein can modulate this interaction, this protein was cloned in the p3H1-σ²⁸ vector under the control of the Met25 promoter. [p3H1-σ²⁸-HP1122]-pP6(β)-transformed cells were almost unable to grow on the selective medium and exhibited very weak β-galactosidase activity (FIG. 3A and B, lanes 2-3). The growth of this strain in non-selective medium (DO-2) was not affected, thus showing that the HP1122 protein effect is not due to toxicity but is rather a direct inhibition of the interaction (FIG. 3A, lanes 2-3). Interestingly, the same inhibitory effect was observed with the SID1122 used as a modulator, encompassing residues 48 to 76 (FIGS. 4A and B, lanes 2-3). The following experiments were used as controls of the specificity of the HP1122 protein: (i) when another protein, such as the SID of HP0419, was added to the same interaction, no inhibitory effect was observed (FIGS. 3A and B, lane 4), (ii) in addition, when HP1122 was added to another interaction, such as the α-β subunits of RNA polymerase, growth and β-galactosidase activities were unaffected thus showing that the inhibition mediated by HP1122 is 28 specific (FIGS. 3A and B, lane 5). Taken together, these results clearly demonstrate that HP1122 specifically prevents interaction between the σ²⁸ and the β subunits of RNA polymerase and confirms that HP1122 is the anti-σ²⁸ factor in H. pylori.

### Example 3

### Inhibition of Transcription from the σ²⁸-dependent Promoter, flaA, by HP1122

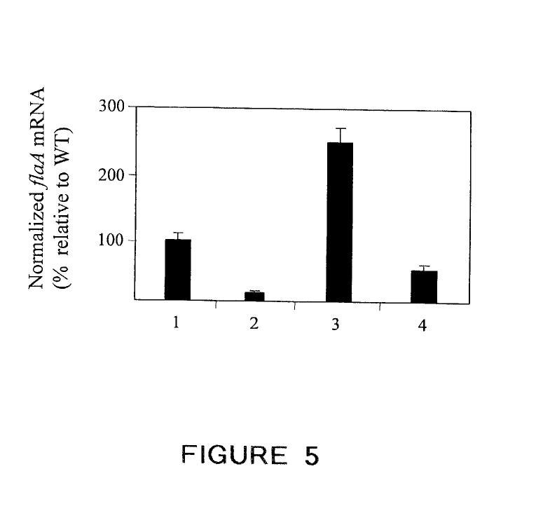

Since HP1122 is suspected to be FlgM, expression of the σ²⁸-regulated promoters is controlled by HP1122 was further investigated. In H. pylori, promoters located upstream of flaG, fliD, fliS (operon fliD) and flaA genes are supposed to be under the control of the σ²⁸ factor (Leying et al., 1992; Kim et al., 1999). To demonstrate the involvement of σ²⁸ and HP1122 in flaA transcription, total RNA was purified from different mutant strains and slot blot hybridization was performed with probes specific of flaA and 16S rRNA as internal control (FIG. 5). RNA slot blot hybridizations.

Total RNAs were extracted from 20 ml of H. pylori culture grown to an optical density at 600 nm (OD600) of 1.0, using the phenol/chloroform extraction procedure, as previously described (Hommais et al., 2001). RNA concentration and purity were determined by OD260 and OD280 measurements. RNA (5 g) was denatured in 350 μl of RNA dilution buffer (1×SSC [0.15 M NaCl plus 0.015 M sodium citrate], 50% formamide and 6.7% formaldehyde) at 68° C. for 15 min and put on ice. The 20×SSC solution (3 M NaCl-0.3 M sodium citrate adjusted to pH 7) was treated with diethylpyrocarbonate. RNA was then applied to Hybond N+ nylon filters (Amersham) with a Minifold I-spot blotter (Schleicher & Schuell). The RNAs were covalently cross-linked to the membrane by UV cross-linking for 10 min.

The flaA (479-bp) and 16S rRNA probes were generated by PCR amplification using oligonucleotides 2564-2565 for flaA (5′-AATGTCGTTTCGGCTTCTGA-3′ (SEQ ID No. 26), and 5′-TAAAAGCCTTMGATATT-3′ (SEQ ID No. 27), respectively) and H276f-H676r for 16S rRNA (5′-CTATGACGGGTATCCGGC-3′ (SEQ ID No. 28) and 5′-ATTCCACCTACCTCTCCCA-3′ (SEQ ID No. 29), respectively). Both DNA probes were labeled with [α-³²P]dCTP (3000 Ci/mmol) using a megaprime labeling kit (Amersham Pharmacia Biotech) and hybridized with the immobilized RNA at 65° C. for 16 h in hybridization buffer (5×SSC, 10% sodium dodecyl sulfate (SDS), 1×Denhardt's reagent and 100 μg.ml⁻¹ sonicated salmon sperm DNA). The membrane was washed three times with 0.5×SSC-0.2% SDS at room temperature and then three times with the same buffer at 65° C. The labeled probes were analyzed and quantified on the STORM (Molecular Dynamics). Experiments were performed in triplicate.

Isogenic mutant fliA of H. pylori N6 was obtained by insertion of a nonpolar kanamycin cassette. FIG. 5 shows that inactivation of the fliA gene resulted in dramatically reduced amount of flaA mRNA as compared to wild-type strain (compare lanes 1 and 2), thus showing that the expression of the flaA promoter is controlled by the σ²⁸ factor of RNA polymerase. In contrast, inactivation of the HP1122 gene elevates expression of the flaA promoter about 2.5-fold as compared to wild-type strain (FIG. 5, compare lanes 1 and 3). This result demonstrates the involvement of HP1122 in negative regulation of flaA transcription. To strengthen the model further, the effect of overexpression of HP1122 on flaA transcription was studied. The stable shuttle vector pRH220cat (Heuermann & Haas, 1998) was used to express the HP1122 protein under the control of the amiE promoter (Skouloubris et al., 1997). In the HP1122-overexpressing N6 strain, the amount of flaA mRNA decreased by a 2-fold factor as compared to the wild-type strain (FIG. 5, compare lanes 1 and 4). All these data support the model in which HP1122 represses transcription of the σ²⁸-dependent promoter, flaA, by inhibiting the activity of σ²⁸.

### Example 4

### Synthesis of Flagellar Organelles is Negatively Regulated by HP1122

It was shown that the flaA expression is dependent on the σ²⁸ factor and is inhibited by HP1122. The FlaA protein is the major species of the flagellar filament and its absence results in truncated flagella, as observed by electron microscopy (Josenhans et al., 1995). To confirm the involvement of σ²⁸ and HP1122 in flagellar assembly, the phenotype of the different wild-type and mutant strains by electron microscopy was examined.

Electron microscopy assay was performed as follows: Cells were briefly washed with PBS, deposited onto formvar coated copper grids and negatively stained with 1% ammonium molybdate in water. The samples were then viewed and photographed.

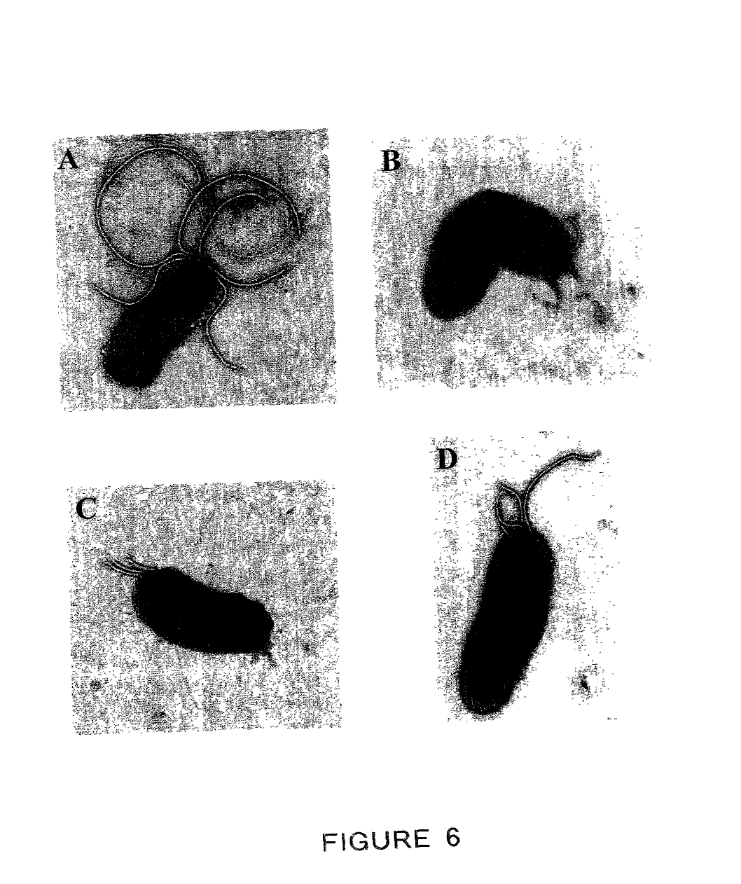

The fliA mutant expresses flagella which were truncated as compared to the wild-type strain (FIG. 6A and B). The aspect of the flagella is similar to the phenotype obtained with a flaA mutant strain (Josenhans et al., 1995) and clearly confirms that the σ²⁸ factor positively controls flagellar biosynthesis and specifically flaA transcription. The effect of overexpression of HP1122 on flagellar formation was then undertaken. This overexpression in H. pylori results predominantly in truncated flagella as compared to wild-type strain (FIGS. 6A and C), thus showing that HP1122 negatively regulates biosynthesis of flagella biosynthesis. Interestingly, SID1122, corresponding to the last 29 residues of HP1122, was overexpressed by the same plasmid in H. pylori and was also shown to affect flagellar synthesis (FIGS. 6A and D). All these data support the model in which HP1122 acts as an anti-σ²⁸ factor by inhibiting the activity of σ²⁸ and thus interferes with flagellar biosynthesis. Finally, inactivation of the HP1122 gene exhibited no significant differences with the wild-type strain in the number and size of flagellar appendages. The absence of additional or longer flagella in this mutant strain could be explained by the involvement of another σ factor, σ⁵⁴, in regulation of flagellar biosynthesis (Suerbaum et al., 1993; Spohn & Scarlato, 1999). The activity of σ⁵⁴, which is independent of HP1122, could be the limiting step in flagellar assembly.

### Example 5

### Identification of the Anti-σ²⁸ Factor of Campylobacter jejuni

By performing BLAST search between the HP1122 protein sequence (SEQ ID NO:6) and the genome sequence of the food-born pathogen Campylobacter jejuni (Parkhill et al., 2000), it was concluded that the hypothetical protein Cj1464 of an unknown function is the anti-σ²⁸ factor of Campylobacter jejuni.

The Cj1464 amino acid sequence is: MINPIQQSYV ANTALNTNRI DKETKTNDTQ KTENDKASKI AEQIKNGTYK IDTKATAMI ADSLI (SEQ ID NO:9).

### Example 6

### Identification of the Anti-σ²⁸ Factor of Pseudomonas aeruginosa

By performing BLAST search between the HP1122 protein sequence (SEQ ID NO:6) and the genome sequence of the pathogen Pseudomonas aeruginosa (Stover et al., 2000), it was concluded that the hypothetical protein PA3351 of unknown function is the anti-σ²⁸ factor of Pseudomonas aeruginosa.

The PA3351 amino acid sequence is: MVIDFNRLNP GSTPATTGRT GSTMGRPDA TGADKAGQM TSAPKSGESV QISETAQNMQ KVTDQLQTLP VVWDNDKVARI KQAIADGTYQ VDSERVASKL LDFESQR (SEQ ID NO:10).

All of the non-patent websites, as well as all patent publications used throughout the specification are hereby incorporated by reference.

While the invention has been described in terms of various preferred embodiments, the skilled artisan will appreciate that various modifications, substitutions, omissions and changes may be made without departing from the scope thereof. Accordingly, it is intended that the scope of the present invention be limited solely by the scope of the following claims, including equivalents thereof.

### BIBLIOGRAPHY

Biocca et al., Embo. J. 9(1):101-8 (1990).

Blum et a., Proc Natl. Acad. Sci. USA 97(5):2241-6 (2000).

Chadsey et al., Genes Dev 12:3123-36 (1998).

Covacci et al., Proc Natl Acad Sci USA 90:5791-5 (1993).

Coveret et al., J Biol Chem 269:10566-73 (1994).

Cussac et al., J Bacteriol 174:2466-73 (1992).

Daughdrill et al., Nat Struct Biol 4:285-91 (1997).

Eaton et al., J Med Microbiol 37:123-7 (1992).

Edwards et al., Anal Biochem 246:1-6 (1997).

Gillen et al., J Bacteriol 173:6453-9 (1991a).

Gillen et al., J Bacteriol 173:2301-10 (1991b).

Goldspiel et al., Clin Pharm 12:488-505 (1993).

Haas et al., Mol Microbiol 8:753-60 (1993).

Heuermann et al., Mol Gen Genet 257:519-28 (1998).

Hommais et al., Mol. Microbiol. In press (2001)

Hughes et al., Science 262:1277-80 (1993).

Hughes et al., Annu Rev Microbiol 52:231-86 (1998).

Jacoby et al., Gene Ther 4:281-3 (1997).

Josenhans et al., J Bacteriol 177:3010-20 (1995).

Kerjan et al., Nucleic Acids Res 14:7861-71 (1986).

Kim et al., J Bacteriol 181:6969-76 (1999).

Kochanek, S. Hum Gene Ther 10:2451-9 (1999).

Kutsukake, K. Mol Gen Genet 243:605-12 (1994).

Labigne et al., Res Microbiol 143:15-26 (1992).

Labigne-Roussel et al., J. Bacteriol. 170:1704-8 (1998).

Leying et al., Mol Microbiol, 6:2863-74 (1992).

Lonetto et al. J Bacteriol, 174:3843-9 (1992).

MacBeath et al., Science, 289:1760-3 (2000).

Macnab R. M., Flagella and motility, in Escherichia coli and Salmonella: cellular and

molecular biology, in: Neidhardt, et al. (Eds), ASM Press, Washington, DC, 96, pp.123-145.

Marshall et al., Lancet 1:1311-5 (1984).

McGowan et al., Gastroenterology 110:926-38 (1996).

Menard et al., Antimicrob Agents Chemother 37:78-83 (1993).

Miller et al., Methods Enzymol 217:581-99 (1993).

Needleman et al., J Mol Biol 48:443-53 (1970).

Ohnishi et al., Mol Gen Genet 221:139-47 (1990).

Ohnishi et a., Microbiol 6:3149-57 (1992).

O'Toole et al., Microbes Infect 2:1207-14 (2000).

Parkhill et al., Nature 403:665-8 (2000).

Pearson et al., Proc Natl Acad Sci U S A 85:2444-8 (1988).

Rain et al., Nature 409:211-5 (2001).

Reynolds et al., Mol Med Today 5:25-31 (1999).

Robbins et al., Pharmacol Ther 80:35-47 (1998).

Sambrook et al., Molecular cloning: A Laboratory Manual. 2ed. Cold Spring Harbor Laboratory, Cold Spring Harbor, N.Y. (1989).

Skouloubris et al., Mol. Microbiol. 25:989-98 (1997).

Smith et al., J Theor Biol 91:379-80 (1981).

Spohn et al., VJ Bacteriol 181:593-9 (1999).

Stover et al., Nature; 406(6799):959-64 (2000).

Brody et al., Trends Microbiol, 3:168-70; discussion 170-1 (1995).

Suerbaum et al., J Bacteriol 175:3278-88 (1993).

Szabo et al., Curr Opin Struct Biol 5:699-705 (1995).

Telford et al., J Exp Med 179:1653-58 (1994).

Tirode et al., J Biol Chem 272:22995-9 (1997).

Tomb et al. [published erratum appears in Nature Sep. 25, 1997;389(6649):412] Nature 388:539-47 (1997).

Tummuru et al., Infect Immun 61:1799-809 (1993).

Wu et al., [published erratum appears in J Biol Chem Jan. 5, 1998;263(1):588]. J Biol Chem 262:4429-32 (1987).

Zijistra et al., Nature 342:435-8 (1989).

What is claimed is:

1. An isolated polypeptide comprising the sequence of SEQ ID NO:2 or NO:4, or a fragment or variant thereof.

2. A fragment of said polypeptide of claim 1, wherein said fragment is a functional fragment.

3. A variant of said polypeptide of claim 1, wherein said variant is a functional variant.

4. An isolated polynucleotide comprising the sequence of SEQ ID NO:1 or NO:3 or a fragment or variant thereof.

5. A fragment of said polypeptide of claim 4, wherein said fragment is a functional fragment.

6. A variant of said polypeptide of claim 4, wherein said variant has a 96% sequence identity to SEQ ID NO:1 or 3.

7. An Helicobacter pylori anti-σ²⁸ factor comprising a polypeptide sequence of SEQ ID NO:6, or a fragment or variant thereof.

8. A fragment of said Helicobacter pylori anti-σ²⁸ factor polypeptide of claim 7, wherein said fragment is a functional fragment.

9. A variant of said Helicobacter pylori anti-σ²⁸ factor polypeptide of claim 7, wherein said variant has a 96% sequence identity to SEQ ID NO:6.

10. An Helicobacter pylori anti-σ²⁸ factor comprising a polynucleotide sequence of SEQ ID NO:5 or a fragment or variant thereof.

11. A fragment of said Helicobacter pylori anti-σ²⁸ factor polynucleotide of claim 10, wherein said fragment is a functional fragment.

12. A variant of said Helicobacter pylori anti-σ²⁸ factor polynucleotide of claim 10, wherein said variant has 96% sequence identity to SEQ ID NO:5.

13. An expression vector comprising the polynucleotide of claim 4.

14. A recombinant host cell comprising the expression vector of claim 13.

15. A complex of at least two interacting polypeptides comprising HP1122 protein (SEQ ID NO:6) and HP1032 protein (SEQ ID NO:8), HP1122 protein (SEQ ID NO:6) and SID1032 (SEQ ID NO:4), SID1122 (SEQ ID NO:2) and HP1032 protein (SEQ ID NO:8) or SID1122 (SEQ ID NO:2) and SID1032 (SEQ ID NO:4).

16. A complex of at least two interacting polypeptides comprising:
a polypeptide having at least 95% amino acid identity with SEQ ID NO:6 and
a polypeptide having at least 95% amino acid identity with SEQ ID NO:8; or
a polypeptide having at least 95% amino acid identity with SEQ ID NO:6 and
a polypeptide having at least 95% amino acid identity with SEQ ID NO:4; or
a polypeptide having at least 95% amino acid identity with SEQ ID NO:2 and
a polypeptide having at least 95% amino acid identity with SEQ ID NO:8; or
a polypeptide having at least 95% amino acid identity with SEQ ID NO:2 and
a polypeptide having at least 95% sequence identity with SEQ ID NO:4.

17. A set of at least two polynucleotides selected from the group comprising:
a first polynucleotide encoding HP1122 (SEQ ID NO:6) and
a second polynucleotide encoding HP1032 (SEQ ID NO:8);
a first polynucleotide encoding HP1122 (SEQ ID NO:6) and
a second polynucleotide encoding SID1032 (SEQ ID NO:4);
a first polynucleotide encoding SID1122 (SEQ ID NO:2) and
a second polynucleotide encoding HP1032 (SEQ ID NO:8);
a first polynucleotide encoding SID1122 (SEQ ID NO:2) and
a second polynucleotide encoding SID1032 (SEQ ID NO:4), and combinations thereof.

18. A set of at least two polynucleotides selected from the group comprising:
a first polynucleotide of sequence SEQ ID NO:5 and a second polynucleotide of SEQ ID NO:7;
a first polynucleotide of sequence SEQ ID NO: 5 and a second polynucleotide of SEQ ID NO:3;
a first polynucleotide of sequence SEQ ID NO:1 and a second polynucleotide of SEQ ID NO:7; and
a first polynucleotide of sequence SEQ ID NO:1 and a second polynucleotide of SEQ ID NO:3, and combinations thereof.

19. A set of at least two polynucleotides selected from the group comprising:
a first polynucleotide having at least 70% nucleic acid identity with sequence SEQ ID NO.5 and a second polynucleotide having at least 70% nucleic acid identity with sequence SEQ ID NO:7;
a first polynucleotide having at least 70% nucleic acid identity with sequence SEQ ID NO:5 and a second polynucleotide having at least 70% nucleic acid identity with sequence SEQ ID NO:3;
a first polynucleotide having at least 70% nucleic acid identity with sequence SEQ ID NO:1 and a second polynucleotide having at least 70% nucleic acid identity with sequence SEQ ID NO:7;
a first polynucleotide having at least 70% nucleic acid identity with sequence SEQ ID NO:1 and a second polynucleotide having at least 70% nucleic acid identity with sequence SEQ ID NO:3 and combinations thereof.

20. An expression vector comprising the set of at least two polynucleotides of claim 17.

21. An expression vector comprising the set of at least two polynucleotides of claim 18.

22. An expression vector comprising the set of at least two polynucleotides of claim 19.

23. A recombinant host cell comprising the expression vector of claim 20.

24. A recombinant host cell comprising the expression vector of claim 21.

25. A recombinant host cell comprising the expression vector of claim 22.
